(19) United States
(12) Patent Application Publication
(10) Pub. No.: US 2025/0296979 A1
(43) Pub. Date: Sep. 25, 2025

(54) Pharmaceutical Composition and Method of Using Same

(71) Applicant: VEGENICS PTY LIMITED, South Yarra, Victoria (AU)

(72) Inventors: Michael GEROMETTA, South Yarra, Victoria (AU); Angus TESTER, South Yarra, Victoria (AU); OLga BUCZEK, South Yarra, Victoria (AU); Ru CAO, South Yarra, Victoria (AU)

(21) Appl. No.: 19/104,726

(22) PCT Filed: Sep. 1, 2023
(86) PCT No.: PCT/AU2023/050850

§ 371 Date: Feb. 19, 2025

Related U.S. Application Data
(60) Provisional application No. 63/374,366, filed on Sep. 1, 2022.

(51) Int. Cl. C07K 14/71; A61K 38/00

(52) U.S. Cl. CPC C07K 14/71; A61K 38/00

(57) ABSTRACT

Provided herein are pharmaceutical compositions comprising an active agent which is a soluble VEGFR-3 trap molecule. Also provided herein are therapeutic methods and uses involving the pharmaceutical compositions, in particular ocular diseases and disorders, and port devices comprising the pharmaceutical compositions.

This patent application claims priority from U.S. provisional patent application No. 63/374,366 filed on 1 Sep. 2022, the entire contents of which is incorporated herein by this reference.

### FIELD

The present disclosure relates to pharmaceutical compositions comprising an active agent which is a soluble VEGFR-3 trap molecule. The disclosure also relates to therapeutic methods and uses involving the pharmaceutical compositions, in particular ocular diseases and disorders, and to port devices comprising the pharmaceutical compositions.

### BACKGROUND

Loss or deterioration of eyesight is an extremely debilitating condition that can have profound effects on an individual's quality of life. Age-related macular degeneration (AMD) is the leading cause of severe vision impairment in older people. The “wet” form of the disorder develops when abnormal blood vessels grow into the macular, which leak blood or fluid that leads to scarring of the macula and loss of vision. A further severe ocular disorder associated with leaking blood vessels is diabetic macular edema (DME), in which leaking fluid accumulates in the macula arising from damage to blood vessels in individuals having diabetic retinopathy.

Current therapies for AMD and DME include treatment with laser therapy (e.g. laser photocoagulation), and administration of medicines including ranibizumab (Lucentis®), aflibercept (Eylea®, Zaltrap®), brolucizumab (Beovu®), and corticosteroids such as triamcinolone.

Vascular endothelial growth factor (VEGF) proteins and their receptors play important roles in both vasculogenesis, the development of the embryonic vasculature from early differentiating endothelial cells, angiogenesis, the process of forming new blood vessels from pre-existing ones, and lymphangiogenesis, the process of forming new lymph vessels. Dysfunction of the endothelial cell regulatory system is also a key feature of cancer and a number of other diseases associated with abnormal vasculogenesis, angiogenesis and lymphangiogenesis.

Therapies directed to blockade of VEGF/PDGF signalling through their receptors have been approved for the therapy of eye conditions including AMD and DME, as well as for cancers. For example, aflibercept, referred to above, is an inhibitor of VEGF consisting of portions of human VEGF receptor 1 and 2 extracellular domains fused to the Fc portion of human IgG1. It acts by binding to circulating VEGF-A and VEGF-B as well as placental growth factor (PlGF), which normally bind to VEGFR-1 and VEGFR-2, and thus is a VEGFR-1/VEGFR-2 trap molecule.

A further therapy in development for the treatment of eye conditions is OPT-302, a VEGFR-3 trap molecule containing portions of human VEGF receptor 3 extracellular domain which is soluble in fluids such as blood and plasma, and binds circulating VEGF proteins that normally being bind to VEGFR-3, i.e. VEGF-C and VEGF-D. OPT-302 has completed a phase 2b clinical trial in wet age-related macular degeneration (wet-AMD) and a phase 2a clinical trial for DME, and phase 3 clinical trials for wet-AMD are ongoing. Soluble VEGFR-3 trap molecules such as OPT-302 are described in, for example, WO2014/124487 and WO2015/123715, the entire contents of which are incorporated herein by reference.

However, drug discovery and development is a lengthy and complex process, and following identification of the therapeutic agent, there can often be significant obstacles to bringing the medicine to market and gaining approval to treat patients. For example, there can be significant challenges in developing pharmaceutical formulations of active agents which provide the required properties, e.g. safety, having acceptable stability of the active, acceptable stability of the formulation, retaining sufficient activity over time, being convenient to administer and avoiding administration site reactions. For some therapeutic agents, despite efforts to identify a suitable formulation, special storage conditions may be required to achieve an acceptable lifetime of the formulation, such as low or ultra-low temperature conditions.

There remains a need for further pharmaceutical products to treat conditions such as wet-AMD and DME. There also remains a need for formulations of soluble VEGFR-3 trap molecules such as OPT-302 which provide good properties, for example in relation to storage stability.

It is to be understood that, if any prior art publication is referred to herein, such reference does not constitute an admission that the publication forms a part of the common general knowledge in the art, in Australia or any other country.

### SUMMARY OF THE DISCLOSURE

OPT-302, a soluble VEGFR-3 trap molecule, has a tendency to form dimers when present in some aqueous formulations, associated with a loss of purity and binding activity. For example, an aqueous composition of OPT-302 which was developed was found to form high levels of OPT-302 dimers rapidly, and needs to be stored at very low temperatures, e.g. −20° C., to achieve an acceptable lifetime.

Research has now identified a pharmaceutical formulation of OPT-302 which provides unexpectedly good stability properties, and does not require storage at −20° C. in order to achieve an acceptable product lifetime.

Accordingly, in a first aspect, there is provided an aqueous pharmaceutical composition, comprising:
an active agent which is a soluble VEGFR-3 trap molecule, the active agent being present at a concentration in the range of from 5 mg/mL to 250 mg/mL;
   trehalose;
   a buffer; and
   water;
wherein the pH of the aqueous pharmaceutical composition is in the range of from 6.5 to 8.0;
and wherein the pharmaceutical composition comprises trehalose in a concentration of at least 7.0% w/v and/or the pharmaceutical composition does not contain added sodium chloride.

In some embodiments, the pharmaceutical composition does not contain added sodium chloride.

In some embodiments, the pharmaceutical composition comprises trehalose in a concentration of at least 7.0% w/v.

In some embodiments, trehalose is present at a concentration of up to 20% w/v. In some embodiments, trehalose is present at a concentration of from 8.5% w/v to 15% w/v. In some embodiments, the composition comprises about 10.9% w/v trehalose.

In some embodiments, the soluble VEGFR-3 trap molecule comprises a ligand binding polypeptide fused to an immunoglobulin constant domain fragment, the ligand binding polypeptide comprising immunoglobulin-like domains 1-3 of the extracellular domain of human VEGFR-3 and optionally having one or more modifications in an N-glycan region of the extracellular domain.

In some embodiments, the ligand binding polypeptide comprises the amino acid sequence defined by positions 25-329 of SEQ ID NO: 1, with the proviso that positions of the polypeptide corresponding to positions 104-106 of SEQ ID NO: 1 are not identical to N-X-S or N-X-T; wherein the ligand binding polypeptide retains four N-glycosylation sequon sites corresponding to positions 33-35 of SEQ ID NO: 1, positions 166-168 of SEQ ID NO: 1, positions 251-253 of SEQ ID NO: 1, and positions 299-301 of SEQ ID NO: 1, and is glycosylated at said four N-glycosylation sequon sites.

In some embodiments, the immunoglobulin constant domain fragment comprises the amino acid sequence defined by positions 99-330 of SEQ ID NO: 2.

In some embodiments, the soluble VEGFR-3 trap molecule has the amino acid sequence set forth in any of SEQ ID NOs: 3-6, or has an amino acid sequence as defined by positions 1-536 of SEQ ID NO: 3, or has an amino acid sequence as defined by positions 1-536 of SEQ ID NO: 4, or has an amino acid sequence as defined by positions 1-546 of SEQ ID NO: 5, or has an amino acid sequence as defined by positions 1-546 of SEQ ID NO: 6.

In some embodiments, the ligand binding polypeptide comprises the amino acid sequence defined by positions 25-329 of SEQ ID NO: 1; wherein the ligand binding polypeptide retains five N-glycosylation sequon sites corresponding to positions 33-35 of SEQ ID NO: 1, positions 104-106 of SEQ ID NO: 1, positions 166-168 of SEQ ID NO: 1, positions 251-253 of SEQ ID NO: 1, and positions 299-301 of SEQ ID NO: 1, and is glycosylated at said five N-glycosylation sequon sites.

In some embodiments, the immunoglobulin constant domain fragment comprises the amino acid sequence defined by positions 99-330 of SEQ ID NO: 2.

In some embodiments, the soluble VEGFR-3 trap molecule has the amino acid sequence set forth in SEQ ID NO: 7, or has an amino acid sequence as defined by positions 1-547 of SEQ ID NO: 7.

In some embodiments, the active agent is present at a concentration of up to 120 mg/mL. In some embodiments, the active agent is present at a concentration of about 40 mg/mL, or about 80 mg/mL, or about 120 mg/mL.

In some embodiments the pH of the composition is in the range of from 7.2 to 7.8. In some embodiments, the pH of the composition is about 7.5.

In some embodiments, the buffer is a sodium phosphate. In some embodiments, the buffer is present in a concentration in the range of from 5 mM to 100 mM. In some embodiments, the buffer is present in a concentration in the range of up to 50 mM. In some embodiments, the buffer is present in a concentration of about 10 mM.

In some embodiments, the composition comprises a surfactant. In some embodiments, the surfactant is polyoxyethylene (20) sorbitan monolaurate or polyoxyethylene (20) sorbitan monooleate. In some embodiments, the surfactant is present at a concentration in the range of from 0.005% to 0.2% w/v. In some embodiments, the surfactant is present at a concentration of about 0.01% w/v.

In some embodiments, the composition has an osmolality in the range of from 300 mOsm/kg to 1000 mOsm/kg. In some embodiments, the composition has an osmolality in the range of from 350 mOsm/kg. In some embodiments, the composition has an osmolality in the range of from 400 mOsm/kg. In some embodiments, the composition has an osmolality in the range of from 400 mOsm/kg to 600 mOsm/kg.

In some embodiments, the composition is substantially free of sodium chloride.

In some embodiments, the composition does not contain an additional sugar.

In some embodiments, the composition does not contain an additional tonicity modifier.

In some embodiments, the composition essentially consists of an active agent in a concentration of about 40 mg/ml, which is a soluble VEGFR-3 trap molecule that comprises a ligand binding polypeptide fused to an immunoglobulin constant domain fragment, the ligand binding polypeptide comprising immunoglobulin-like domains 1-3 of the extracellular domain of human VEGFR-3 and optionally having one or more modifications in an N-glycan region of the extracellular domain;
trehalose in a concentration of about 10.9% w/v;
sodium phosphate in a concentration of about 10 mM;
polyoxyethylene (20) sorbitan monolaurate in a concentration of about 0.01% w/v; and
   water;
wherein the pH of the aqueous pharmaceutical composition is about 7.5.

In a further aspect, there is provided a lyophilised pharmaceutical composition for reconstitution, comprising:
an active agent which is a soluble VEGFR-3 trap molecule;
   trehalose; and
   a buffer;
wherein the weight ratio of trehalose to active agent is in the range of from 1:3 to 40:1.

In some embodiments, the weight ratio of trehalose to active agent is in the range of from 1:1 to 7.5:1, from 1:1 to 5:1, or from 2.1:1 to 4.5:1. In some embodiments, the weight ratio of trehalose to active agent is about 2.7:1.

In some embodiments, the buffer is a sodium phosphate. In some embodiments, the buffer is a sodium phosphate, and the weight ratio of sodium phosphate to active agent is in the range of from 1:3 to 1:1000, or from 1:3 to 1:200, or from 1:5 to 1:100. In some embodiments, the buffer is a sodium phosphate, and the weight ratio of sodium phosphate to active agent is about 0.03:1.

In some embodiments the composition comprises a surfactant. In some embodiments, the surfactant is polyoxyethylene (20) sorbitan monolaurate or polyoxyethylene (20) sorbitan monooleate.

In a further aspect, there is also provided a reconstituted pharmaceutical composition, wherein the pharmaceutical composition is obtained by admixing a lyophilised pharmaceutical composition as defined herein with an aqueous diluent.

In some embodiments of the aspects defined above, the pharmaceutical composition is formulated for intravitreal injection.

In a further aspect, there is provided a method of inhibiting neovascularisation in a subject, comprising administering to the subject an effective amount of a pharmaceutical composition as defined herein.

In a further aspect there is provided a method of treating and/or preventing a disease or disorder associated with aberrant neovascularisation, angiogenesis and/or lymphangiogenesis in a subject, comprising administering to the subject an effective amount of a pharmaceutical composition as defined herein.

In a further aspect there is provided use of a VEGF-C and/or VEGF-D trap molecule or a salt thereof for the manufacture of a pharmaceutical composition as defined herein, for the treatment and/or prevention of a disease or disorder associated with aberrant neovascularisation, angiogenesis and/or lymphangiogenesis.

In a further aspect there is provided a pharmaceutical composition as defined herein, for use in the treatment and/or prevention of a disease or disorder associated with aberrant neovascularisation, angiogenesis and/or lymphangiogenesis.

In some embodiments of the methods, uses, and pharmaceutical compositions for use,
the disease or disorder is an ocular disease or disorder. In some embodiments of the methods, uses, and pharmaceutical compositions for use, the ocular disease or disorder is selected from the group consisting of macular degeneration, diabetic retinopathy, macular edema, retinal vein occlusion and macular telangiectasia. In some embodiments of the methods, uses, and pharmaceutical compositions for use, the ocular disease or disorder is wet age-related macular degeneration. In some embodiments of the methods, uses, and pharmaceutical compositions for use, the ocular disease or disorder is diabetic macular edema. In some embodiments of the methods, uses, and pharmaceutical compositions for use, the pharmaceutical composition is administered in combination with a further active agent. In some embodiments of the methods, uses, and pharmaceutical compositions for use, the further active agent is an anti-VEGF-A agent or an anti-VEGF-B agent. In some embodiments of the methods, uses, and pharmaceutical compositions for use, the further active agent is selected from the group consisting of ranibizumab, aflibercept, bevacizumab and brolucizumab.

In some embodiments of the methods, uses, and pharmaceutical compositions for use,
the pharmaceutical composition is administered intravitreally.

In some embodiments of the methods, uses, and pharmaceutical compositions for use,
the pharmaceutical composition is administered using a port device which is implanted in an eye, which port device comprises a reservoir for the pharmaceutical composition, and permits controlled release of active agent into the vitreous of the eye.

In a further aspect, there is also provided a port device for implantation in an eye, the port device comprising a reservoir containing a pharmaceutical composition as defined herein, and wherein the port device permits controlled release of active agent into the vitreous of the eye.

In some embodiments, the port device comprises a semipermeable membrane that permits passive diffusion of active agent into the vitreous of the eye.

In some embodiments, the port device comprises a septum which permits refilling of the reservoir with additional pharmaceutical composition using a needle.

### BRIEF DESCRIPTION OF THE DRAWINGS

FIG. 1 shows a chart showing the results of stability studies for OPT-302 compositions according to the present disclosure and comparative compositions. The % level formation of high molecular weight species of active agent over time was determined for compositions at 37° C.

FIG. 2 shows a chart showing the results of stability studies for OPT-302 compositions according to the present disclosure. The % level formation of monomer of active agent over time was determined for compositions at 25° C.

FIG. 3 shows a chart showing the results of stability studies for OPT-302 compositions according to the present disclosure. The % level formation of monomer of active agent over time was determined for compositions at 5° C.

### DETAILED DESCRIPTION

### Definitions

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps, unless otherwise required by context.

As used herein, the term “and/or”, e.g. “X and/or Y”, shall be understood to mean either or both of “X and Y” and “X or Y” and shall be taken to provide explicit support for both meanings or for either meaning.

As used herein, the term about, unless stated to the contrary, refers to +/−10%, more preferably +/−5%, of the designated value.

As used herein, the terms “a”, “an” and “the” include both singular and plural aspects, unless the context clearly indicates otherwise.

As used herein, the phrase “at least one of”, when used with a list of items, means different combinations of one or more of the listed items may be used and only one of the items in the list may be needed.

As used herein, the term “subject” refers to any organism that is susceptible to a disease or condition. For example, the subject can be an animal, a mammal, a primate, a livestock animal (e.g., sheep, cow, horse, pig), a companion animal (e.g., dog, cat), or a laboratory animal (e.g., mouse, rabbit, rat, guinea pig, hamster). In some embodiments, the subject is a mammal. In some embodiments, the subject is human. In some embodiments, the subject is a non-human animal.

As used herein, the terms “treating” and treatment” include one or more of the following: curing a disease or disorder, reducing the severity of a disease or disorder, preventing or slowing progression of a disease or disorder, and alleviation of symptoms associated with a disease or disorder.

As used herein, the terms “preventing” and “prevention” include one or more of the following: preventing a subject from developing a disease or disorder, delaying the onset of a disease or disorder, and prophylaxis of a disorder or condition.

The term “therapeutically effective amount”, as used herein, refers to a pharmaceutical composition comprising a soluble VEGFR-3 trap molecule being administered in an amount sufficient to treat or prevent the disorder or condition being treated.

The term “identity”, as known in the art, refers to a relationship between the sequences of two or more polypeptide molecules or two or more nucleic acid molecules, as determined by comparing the sequences. In the art, “identity” also means the degree of sequence relatedness of nucleic acid molecules or polypeptides sequences, as the case may be, as determined by the match between strings of two or more nucleotide or two or more amino acid sequences. “Identity” measures the percent of identical matches between the smaller of two or more sequences with gap alignments (if any) addressed by particular a mathematical model of computer program (i.e., “algorithms”). Appropriate algorithms for determining the percent identities of the present disclosure include BLASTP and BLASTN, using the most common and accepted default parameters.

The term “component domain” as used herein to refers to a domain within a ligand binding molecule which is derived from or based on a protein domain within the extracellular portion of a receptor protein. For example, each Ig-domain of VEGFR-3 (D1-D7) constitutes a component domain. Reference herein to a component domain includes both the complete native wildtype domain and also insertions, deletions and/or substitutional variants thereof which substantially retain the functional characteristics of the intact domain. It will be readily apparent to one of skill in the art that numerous variants of the above domains (e.g. Ig-domains) can be obtained which will retain substantially the same functional characteristics as the wild-type domain.

### Active Agent

The pharmaceutical compositions of the present disclosure contain an active agent, which is a soluble VEGFR-3 trap molecule.

Vascular endothelial growth factor receptor 3 (VEGFR-3; previously known as Flt4) is the receptor for VEGF-C and VEGF-D ligands and is found primarily on vascular and lymphatic endothelial cells. It is primarily involved in angiogenesis and lymphangiogenesis. Soluble VEGFR-3 trap molecules find utility in the treatment of disease and disorders associated with neovascularization and/or vascular permeability, aberrant angiogenesis and/or lymphangiogenesis, such as a number of ocular disorders including wet age-related macular degeneration and diabetic macular edema. They also have application in other disease indications associated with aberrant angiogenesis and/or lymphangiogenesis (e.g. cancer).

The term “soluble” as used herein in relation to the active agent, means that the active agent has high enough solubility in biological fluid e.g. blood, plasma, and/or vitreous humour, such that it is available for binding circulating VEGF-C. In some embodiments, the soluble VEGFR-3 trap molecule has a solubility in plasma of at least 1 mg/mL, or at least 2 mg/mL, or at least 5 mg/mL, or at least 10 mg/mL, or at least 20 mg/mL, or at least 30 mg/mL, or at least 40 mg/mL.

The active agent is a VEGFR-3 trap molecule. As referred to herein, a VEGFR-3 trap molecule is a molecule which is capable of binding to circulating VEGF-C and/or VEGF-D. In some embodiments, the soluble VEGFR-3 trap molecule binds human VEGF-C with a KD of about 1 nM or less (e.g., 500 pM, 400 pM, 300 pM, 200 pM, 100 pM, 50 pM, 10 pM or less). In some embodiments, the soluble VEGFR-3 trap molecule binds human VEGF-D with a KD of about 5 nM or less (e.g., 2 nM, 1 nM, 500 pM, 400 pM, 300 pM, 200pM, 100 pM, 50 pM, 10 pM or less).

Binding affinity to VEGF-C and VEGF-D may be determined by any suitable assay. For example binding affinity may be determined using ELISA, or using surface plasmon resonance. Such techniques are described in, for example, WO 2014/124487 A1, the entire contents of which is incorporated herein by reference.

In some embodiments, the soluble VEGFR-3 trap molecule is or comprises a polypeptide.

In some embodiments, the soluble VEGFR-3 trap molecule comprises a ligand binding polypeptide. For example, the ligand binding polypeptide may be or may comprise a fragment of a growth receptor tyrosine kinase extracellular domain (ECD). The fragment may in some embodiments vary from the wildtype sequence in ways that do not eliminate growth factor binding, and the fragment preferably is engineered in ways described herein to improve its properties as a therapeutic for administration to subjects/patients in need.

VEGF-C and D bind with high affinity to, and stimulate phosphorylation of, at least one VEGF receptor (or receptor heterodimer) selected from VEGFR-2 and VEGFR-3. Preferred ligand binding polypeptides do more than simply bind their target growth factors. A preferred ligand binding polypeptide also inhibits the growth factor(s) to which it binds from stimulating phosphorylation of at least one (and preferably all) of the receptor tyrosine kinases to which the growth factor(s) bind. Stimulation of tyrosine phosphorylation is readily measured using in vitro cell-based assays and antibodies.

Ligand binding polypeptides that are “specific” for a particular growth factor are ligand binding molecules that specifically recognize an active form of the growth factor (e.g., a form found circulating in the body). Preferably, the ligand binding polypeptide specifically binds other forms of the growth factors as well. By way of example, VEGF-C (and VEGF-D) is translated as a prepro-molecule with extensive amino-terminal and carboxy-terminal propeptides that are cleaved to yield a “fully processed” form of VEGF-C (or VEGF-D) that binds and stimulates VEGFR-2 and VEGFR-3. Ligand binding polypeptides specific for VEGF-C (or VEGF-D) bind to at least the fully processed form of VEGF-C (or VEGF-D), and preferably also bind to partially processed forms and unprocessed forms.

SEQ ID NO: 1 contains an amino acid sequence for human VEGFR-3, with positions 1-24 of SEQ ID NO: 1 corresponding to a putative signal peptide and position 25 onwards of SEQ ID NO: 1 corresponding to a putative mature form of the receptor lacking a putative signal peptide.

In some embodiments, the soluble VEGFR-3 trap molecule comprises a ligand binding polypeptide comprising a portion of the extracellular domain (ECD) of human VEGFR-3. The ECD of human VEGFR-3 contains 7 immunoglobulin-like domains. Domains 1-3 are involved in ligand binding, and domains 4-7 are involved in structural rearrangements essential for receptor dimerization.

The complete ECD of VEGFRs is not required for ligand (growth factor) binding. The ECD of VEGFR-3 has six intact Ig-like domains and one cleaved Ig-like domain-D5 of VEGFR-3 is cleaved post-translationally into disulfide linked subunits leaving VEGFR-3 (Veikkola, T., et al., Cancer Res. 60:203-212 (2000)). In some embodiments, receptor fragments comprising at least the first three Ig-like domains for this family are sufficient to bind ligand. Soluble receptors capable of binding VEGF-C and VEGF-D, thereby inhibiting VEGF-C or VEGF-D activity or signaling via VEGFR-3, are also disclosed in WO2000/023565, WO2000/021560, WO2002/060950 and WO2005/087808, the disclosures of which are incorporated herein by reference in their entireties. Those soluble receptors, optionally with modifications described herein, are contemplated as soluble VEGFR-3 trap molecules of the present disclosure.

The table below defines approximate boundaries of the Ig-like domains for human VEGFR-3. These boundaries are significant as the boundaries chosen can be used to form ligand binding molecules, and so can influence the binding properties of the resulting constructs.

| | VEGFR-3 SEQ ID NO: 1 |
|---|---|
| | positions |
| D1 | 47-115 |
| D2 | 154-210 |
| D3 | 248-314 |
| D4 | 351-403 |
| D5 | 441-538 |
| D6 | 574-657 |
| D7 | 695-752 |

In some embodiments, the ligand binding polypeptide comprises a portion of the amino acid sequence of at least one of immunoglobulin-like domains 1, 2 and 3 of the ECD of human VEGFR-3.

In some embodiments, the ligand binding polypeptide comprises substantially all or all of the amino acid sequence of at least one of immunoglobulin-like domains 1, 2 and 3 of the ECD of human VEGFR-3.

In some embodiments, the soluble VEGFR-3 trap molecule comprises a ligand binding polypeptide which is a purified or isolated ligand binding polypeptide comprising a first amino acid sequence having at least 80%, or at least 85%, or at least 90%, or at least 92%, or at least 95%, or at least 96%, or at least 97%, or at least 98%, or at least 99% identity to the sequence of amino acids defined by positions 47-115 of SEQ ID NO: 1 or positions 25-115 of SEQ ID NO: 1. The foregoing segments of SEQ ID NO: 1 roughly correspond to or include the first immunoglobulin-like domain of the extracellular domain (ECD) of human VEGFR-3 (“D1 of VEGFR-3”).

In some embodiments, the soluble VEGFR-3 trap molecule comprises a ligand binding polypeptide which is a purified or isolated ligand binding polypeptide comprising an amino acid sequence having at least 80%, or at least 85%, or at least 90%, or at least 92%, or at least 95%, or at least 96%, or at least 97%, or at least 98%, or at least 99% identity to the sequence of amino acids defined by positions 47-115 of SEQ ID NO: 1 or positions 25-115 of SEQ ID NO: 1, with the proviso that positions of the polypeptide corresponding to positions 104-106 of SEQ ID NO: 1 are not identical to N-X-S or N-X-T (X representing any amino acid).

Ig-like domains 1-3 of VEGFR-3 comprises five putative N-glycosylation sites (referred to herein as N1, N2, N3, N4 and N5 sequons of VEGFR-3, respectively). N1 corresponds to amino acids 33-35 of SEQ ID NO: 1; N2 corresponds to amino acids 104-106 of SEQ ID NO: 1; N3 corresponds to amino acids 166-168 of SEQ ID NO: 1; N4 corresponds to amino acids 251-253 of SEQ ID NO: 1 and N5 corresponds to amino acids 299-301 of SEQ ID NO: 1. In some embodiments, a ligand binding molecule described herein comprises a modification in the N2 sequon of the molecule.

In some embodiments, said putative glycosylation sequon at positions 104-106 is eliminated from the amino acid sequence of the ligand binding polypeptide. The term “eliminated” as used in this context means an alteration of the primary amino acid sequence in at least one position (by substitution, deletion or insertion) to destroy the N-X-T sequon motif. In one variation, the amino acid corresponding to position 104 of SEQ ID NO: 1 may be deleted and replaced with another amino acid (such as glutamine, aspartate, glutamate, arginine and lysine).

For example, in some embodiments, the amino acid in the ligand binding molecule corresponding to position 104 of SEQ ID NO: 1 is deleted and replaced with another amino acid. Conservative substitutions are preferred. In some embodiments, the amino acid corresponding to position 104 of SEQ ID NO: 1 is deleted and replaced with an amino acid selected from the group consisting of glutamine, aspartate, glutamate, arginine and lysine. In embodiments where the N2 sequon of SEQ ID NO: 1 is modified as described above, the N1, N3, N4 and N5 sequons of SEQ ID NO: 1 are preferably unaltered in terms of amino acid sequence.

In some embodiments, the soluble VEGFR-3 trap molecule comprises a ligand binding polypeptide which is a purified or isolated ligand binding polypeptide sequence comprising an amino acid sequence having at least 80%, or at least 85%, or at least 90%, or at least 92%, or at least 95%, or at least 96%, or at least 97%, or at least 98%, or at least 99% identical to the sequence of amino acids defined by positions 154-210 of SEQ ID NO:1. The sequence of amino acids defined by positions of the polypeptide corresponding to positions 154-210 roughly corresponds to or includes the second immunoglobulin-like domain of the ECD of human VEGFR-3 (“D2 of VEGFR-3”).

In some embodiments, the soluble VEGFR-3 trap molecule comprises a ligand binding polypeptide which is a purified or isolated ligand binding polypeptide sequence comprising an amino acid sequence having at least 80%, or at least 85%, or at least 90%, or at least 92%, or at least 95%, or at least 96%, or at least 97%, or at least 98%, or at least 99% identical to the sequence of amino acids defined by positions 248-314 of SEQ ID NO: 1. The sequence of amino acids defined by positions of the polypeptide corresponding to positions 248-314 roughly corresponds to or includes the third immunoglobulin-like domain of the ECD of human VEGFR-3 (“D3 of VEGFR-3”).

In some embodiments, the ligand binding polypeptide comprises substantially all or all of the amino acid sequence of immunoglobulin-like domains 1 and 2 of the ECD of human VEGFR-3. In some embodiments, the ligand binding polypeptide comprises substantially all or all of the amino acid sequence of immunoglobulin-like domains 2 and 3 of the ECD of human VEGFR-3. In some embodiments, the ligand binding polypeptide comprises substantially all or all of the amino acid sequence of immunoglobulin-like domains 1, 2 and 3 of the ECD of human VEGFR-3.

In some embodiments, the soluble VEGFR-3 trap molecule comprises a ligand binding polypeptide comprising immunoglobulin-like domains 1-3 of the extracellular domain of human VEGFR-3 and optionally having one or more modifications in an N-glycan region of the extracellular domain.

In some embodiments, the soluble VEGFR-3 trap molecule comprises a ligand binding polypeptide which is a purified or isolated ligand binding polypeptide sequence comprising an amino acid sequence having at least 80%, or at least 85%, or at least 90%, or at least 92%, or at least 95%, or at least 96%, or at least 97%, or at least 98%, or at least 99% identical, or which is identical to the sequence of amino acids defined by positions 25-329 of SEQ ID NO:1.

In some embodiments, the soluble VEGFR-3 trap molecule comprises a ligand binding polypeptide comprising the amino acid sequence defined by positions 25-329 of SEQ ID NO: 1; wherein the ligand binding polypeptide retains five N-glycosylation sequon sites corresponding to positions 33-35 of SEQ ID NO: 1, positions 104-106 of SEQ ID NO: 1, positions 166-168 of SEQ ID NO: 1, positions 251-253 of SEQ ID NO: 1, and positions 299-301 of SEQ ID NO: 1, and is glycosylated at said five N-glycosylation sequon sites.

In some embodiments, the soluble VEGFR-3 trap molecule comprises a ligand binding polypeptide which is a purified or isolated ligand binding polypeptide sequence comprising an amino acid sequence having at least 80%, or at least 85%, or at least 90%, or at least 92%, or at least 95%, or at least 96%, or at least 97%, or at least 98%, or at least 99% identical, or which is identical to the sequence of amino acids defined by positions 25-329 of SEQ ID NO:1, with the proviso that positions of the polypeptide corresponding to positions 104-106 of SEQ ID NO: 1 are not identical to N-X-S or N-X-T (X representing any amino acid).

In some embodiments, the soluble VEGFR-3 trap molecule comprises a ligand binding polypeptide comprising the amino acid sequence defined by positions 25-329 of SEQ ID NO: 1, with the proviso that positions of the polypeptide corresponding to positions 104-106 of SEQ ID NO: 1 are not identical to N-X-S or N-X-T; wherein the ligand binding polypeptide retains four N-glycosylation sequon sites corresponding to positions 33-35 of SEQ ID NO: 1, positions 166-168 of SEQ ID NO: 1, positions 251-253 of SEQ ID NO: 1, and positions 299-301 of SEQ ID NO: 1, and is glycosylated at said four N-glycosylation sequon sites.

In some embodiments, said putative glycosylation sequon at positions 104-106 is eliminated from the amino acid sequence of the ligand binding polypeptide. The term “eliminated” as used in this context means an alteration of the primary amino acid sequence in at least one position (by substitution, deletion or insertion) to destroy the N-X-T sequon motif. In one variation, the amino acid corresponding to position 104 of SEQ ID NO: 1 may be deleted and replaced with another amino acid (such as glutamine, aspartate, glutamate, arginine and lysine).

Constructs that comprise additional Ig-like domains of VEGFR-3, attached in a manner that result in a ligand binding polypeptide, are specifically contemplated. For example, the soluble VEGFR-3 trap molecule may contain portions of, or substantially all or all of, seven Ig-like domains of VEGFR-3.

In embodiments where the ligand binding polypeptide comprises amino acid sequences roughly corresponding to two or more component domains of VEGFR-3, the component domains may be connected directly to each other or may be connected via one or more spacers. Preferably, the component domains are connected by one or more spacers.

For example, the ligand binding polypeptide may optionally include sequence before the most N-terminally positioned Ig-like domain, between the Ig-like domains, and/or after the most C-terminally positioned Ig-like domain.

In one embodiment, the spacer comprises one or more peptide sequences between the component domains which is (are) between 1-100 amino acids, preferably 1-50 amino acids in length. In one embodiment, the spacer between two component domains substantially consists of peptide sequences naturally connected to the component domain in native VEGFR-3.

In embodiments where the ligand binding polypeptide comprises amino acid sequences roughly corresponding to or including contiguous component domains of VEGFR-3 (for example, D1-D2 or D1-D2-D3), the component domains may be connected via one or more spacers comprising one or more peptide sequences between the component domains which is (are) between 1-100 amino acids, preferably 1-50 amino acids in length.

In some embodiments, the spacer between two component domains substantially consists of peptide sequences corresponding to those connecting the respective contiguous component domains in the native VEGFR-3. In some embodiments, the spacer between two contiguous component domains comprises an amino acid sequence at least 80%, or at least 85%, or at least 90%, or at least 92%, or at least 95%, or at least 96%, or at least 97%, or at least 98%, or at least 99% identical, or which is identical, to the sequence of amino acids that connects the contiguous domains in the native VEGFR-3.

In embodiments where the soluble VEGFR-3 trap molecule comprises a ligand binding polypeptide comprises multiple component domains, for example component domains D1, D2 and D3 of VEGFR-3, the component domains may be connected directly to each other or may be connected via one or more spacers. Generally, the term “spacer” means one or more molecules, for example nucleic acids or amino acids, or non-peptide moieties, such as polyethylene glycol or disulfide bridges, which may be inserted between one or more component domains forming a covalent bond. Spacer sequences may be used to provide a desirable site of interest between components for ease of manipulation. A spacer may also be provided to enhance expression of the ligand binding polypeptide from a host cell, to decrease steric hindrance such that the component or group of components may assume its/their optimal tertiary structure and/or interact appropriately with its/their target molecule. For spacers and methods of identifying desirable spacers, see, for example, George et al. (2003) Protein Engineering 15:871-879, herein specifically incorporated by reference. A spacer sequence may include one or more amino acids naturally connected to a receptor component, or may be an added sequence used to enhance expression of the ligand binding polypeptides, provide specifically desired sites of interest, allow component domains to form optimal tertiary structures and/or to enhance the interaction of a component or group of components with its/their target molecule. In one embodiment, the spacer comprises one or more peptide sequences between one or more components which is (are) between 1-100 amino acids, preferably 1-50 amino acids in length. In a preferred embodiment, the spacer between two component domains substantially consists of amino acids naturally connected to the receptor component in the wildtype receptor. In the case of a ligand binding polypeptide comprising multiple component domains from the same receptor which domains are adjacent each other in the native receptor, such as for example D1, D2 and D3 of VEGFR-3, in one embodiment, the domains are connected to each other (e.g. D1 to D2 and D2 to D3) using spacers corresponding to the naturally-occurring amino acid linking sequences.

In some variations, each ligand binding polypeptide is expressed as a fusion with a fusion partner protein, such as an immunoglobulin constant region, and the heterologous fusion partners are linked to form the ligand binding molecule.

In some embodiments, the ligand binding molecule is a polypeptide that comprises a portion of a human VEGFR-3 ECD, wherein the portion binds to one or both of human VEGF-C and human VEGF-D, and comprises at least the first, second and third Ig-like domains of the VEGFR-3 ECD, wherein the amino acid sequence of the ECD fragment of VEGFR-3 is modified from wildtype VEGFR-3 to eliminate the second putative N-linked glycosylation sequon of wildtype VEGFR-3, and wherein the polypeptide lacks VEGFR-3 Ig-like domains 4-7 and preferably any transmembrane domain and preferably any intracellular domain.

Alongside monomeric constructs, the present disclosure also includes multimeric ligand binding constructs comprising two or more ligand binding molecules as described herein, covalently or non-covalently attached to each other to form a dimeric or multimeric structure. In some variations, the attachment occurs between the VEGFR-3-like sequences of the ligand binding polypeptides; in other variations, the attachment occurs between heterologous polypeptides attached to one or both of the VEGFR-3 like sequences.

Reference herein to a ligand binding polypeptide described herein includes reference to variants thereof. In some embodiments, the ligand binding polypeptide is a variant. In other embodiments, the ligand binding polypeptide is not a variant.

VEGFR-3, from which ligand binding polypeptides may be derived, include splice variants and naturally-occurring allelic variations. Allelic variants are well known in the art, and represent alternative forms or a nucleic acid sequence that comprise substitution, deletion or addition of one or more nucleotides, but which do not result in any substantial functional alteration of the encoded polypeptide. Exemplary allelic variants of VEGFR-3 have been reported in the literature, e.g., at http://www.uniprot.org/uniprot/P35916, and include positions 149, 378, 494, 527, and 641 within the ECD. Standard methods can readily be used to generate such polypeptides including site-directed mutagenesis of polynucleotides, or specific enzymatic cleavage and ligation. Similarly, use of peptidomimetic compounds or compounds in which one or more amino acid residues are replaced by a non-naturally-occurring amino acid or an amino acid analog that retain binding activity is contemplated.

Preferably, where amino acid substitution is used, the substitution is conservative, i.e. an amino acid is replaced by one of similar size and with similar charge properties. As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic acid for aspartic acid, or glutamine for asparagine, and the like. Neutral hydrophilic amino acids that can be substituted for one another include asparagine, glutamine, serine and threonine. The term “conservative substitution” also includes the use of a substituted amino acid in place of an unsubstituted amino acid.

Alternatively, conservative amino acids can be grouped as described in Lehninger, (Biochemistry, Second Edition; Worth Publishers, Inc. NY:NY, pp. 71-77 (1975)) as set out in the following:
Non-polar (hydrophobic)
   A. Aliphatic: A, L, I, V, P,
   B. Aromatic: F, W,
   C. Sulfur-containing: M,
   D. Borderline: G.
Uncharged-polar
   A. Hydroxyl: S, T, Y,
   B. Amides: N, Q,
   C. Sulfhydryl: C,
   D Borderline: G.
Positively Charged (Basic): K, R, H.
Negatively Charged (Acidic): D, E.

The soluble VEGFR-3 trap molecule may for example contain a fusion partner (such as, for example, a heterologous peptide), e.g. to impart desired characteristics (such as, for example, increase the serum half-life, increase the solubility in an aqueous medium and/or enable targeting to a specific cell population, e.g., tumor cells or retinal cells).

In some embodiments, the fusion partner is any heterologous component that enhances the functionality of the ligand binding polypeptide. Thus, for example, a fusion partner may increase the solubility, modulate the clearance, facilitate targeting of particular cell or tissue types, enhance the biological activity, aid the production and/or recovery, enhance a pharmacological property or enhance a pharmacokinetic (PK) profile of the ligand binding polypeptide. With regards to enhancing the PK profile, this may be achieved by, for example, enhancing the serum half-life, tissue penetrability, lack of immunogenicity or stability of the ligand binding molecule. In some embodiments, a fusion partner is selected from the group consisting of a multimerizing component, a serum protein or a molecule capable of binding a serum protein.

In some embodiments, the fusion component comprises an immunoglobulin-derived domain from, for example, human IgG, Ig or IgA.

In some variations, the soluble VEGFR-3 trap molecule comprises an immunoglobulin constant domain or fragment thereof. In some embodiments, the soluble VEGFR-3 trap molecule comprises a human immunoglobulin G domain or fragment thereof. The amino acid sequence of human immunoglobulin 1 heavy chain constant domain is set out in SEQ ID NO: 2. In some embodiments, the immunoglobulin constant domain fragment comprises the amino acid sequence defined by positions 99-330 of SEQ ID NO: 2.

In some embodiments, the soluble VEGFR-3 trap molecule comprises an immunoglobulin constant domain fragment which comprises an amino acid sequence having at least 80%, or at least 85%, or at least 90%, or at least 92%, or at least 95%, or at least 96%, or at least 97%, or at least 98%, or at least 99% identical, or which is identical to the sequence of amino acids defined by positions 99-330 of SEQ ID NO:2.

In some embodiments, the immunoglobulin constant domain fragment may have a C-terminal amino acid residue omitted. For example, in some embodiments, the immunoglobulin constant domain fragment has the amino acid sequence defined by positions 99-329 of SEQ ID NO: 2.

The amino acid sequence derived from the immunoglobulin may be linked to the C-terminus or to the N-terminus of the ligand binding polypeptide, preferably to the C-terminus. Cells transfected with DNA encoding the immunoglobulin light chain fusion protein and the immunoglobulin heavy chain fusion protein express heavy chain/light chain heterodimers containing each a ligand binding polypeptide. Both ligand binding polypeptides advantageously comprise a native or heterologous signal peptide when initially synthesized, to promote secretion from the cell, but the signal sequence ma for example be cleaved upon secretion. Variations of any of the foregoing embodiments that include the signal peptide are contemplated. The native signal peptide of human VEGFR-3 comprises residues 1-24 of SEQ ID NO: 1. Numerous other signal peptide proteins are taught in the literature.

In some embodiments, the soluble VEGFR-3 trap molecule comprises a ligand binding polypeptide which is fused (as a single polypeptide chain) to the Fc portion of human immunoglobulin G (IgG).

In some embodiments, the soluble VEGFR-3 trap molecule comprises a ligand binding polypeptide which is fused (as a single polypeptide chain) to the Fc portion of human immunoglobulin G (IgG), in which there is a single amino acid substitution in the second Ig-like domain to remove an N-glycosylation site.

In some embodiments, a ligand binding polypeptide as described herein optionally comprises a linker connecting the fusion partner, such as, for example, a heterologous peptide to the ligand binding polypeptide, such as the factor Xa linker sequence DPIEGRGGGGG (SEQ ID NO: 8). In other embodiments, the ligand binding molecule comprises a polypeptide in which a C-terminal amino acid of the ligand binding polypeptide is directly attached to an N-terminal amino acid of the heterologous peptide fusion partner by a peptide bond. In some embodiments, the ligand binding polypeptide and the heterologous peptide are attached (directly or through a linker polypeptide) by amide bonding to form a single polypeptide chain.

In some embodiments, the soluble VEGFR-3 trap molecule is OPT-302. OPT-302 has the amino acid sequence set forth in SEQ ID NO: 3.

In some embodiments, the soluble VEGFR-3 trap molecule comprises the amino acid sequence set forth in SEQ ID NO: 3. In some embodiments, the soluble VEGFR-3 trap molecule has an amino acid sequence consisting of the amino acid sequence set forth in SEQ ID NO: 3. In some embodiments, the soluble VEGFR-3 trap molecule has an amino acid sequence defined by positions 1-536 of SEQ ID NO: 3.

In some embodiments, the soluble VEGFR-3 trap molecule comprises the amino acid sequence set forth in SEQ ID NO: 4. In some embodiments, the soluble VEGFR-3 trap molecule has an amino acid sequence consisting of the amino acid sequence set forth in SEQ ID NO: 4. In some embodiments, the soluble VEGFR-3 trap molecule has an amino acid sequence defined by positions 1-536 of SEQ ID NO: 4.

In some embodiments, the soluble VEGFR-3 trap molecule comprises the amino acid sequence set forth in SEQ ID NO: 5. In some embodiments, the soluble VEGFR-3 trap molecule has an amino acid sequence consisting of the amino acid sequence set forth in SEQ ID NO: 5. In some embodiments, the soluble VEGFR-3 trap molecule has an amino acid sequence defined by positions 1-546 of SEQ ID NO: 5.

In some embodiments, the soluble VEGFR-3 trap molecule comprises the amino acid sequence set forth in SEQ ID NO: 6. In some embodiments, the soluble VEGFR-3 trap molecule has an amino acid sequence consisting of the amino acid sequence set forth in SEQ ID NO: 6. In some embodiments, the soluble VEGFR-3 trap molecule has an amino acid sequence defined by positions 1-546 of SEQ ID NO: 6.

In some embodiments, the soluble VEGFR-3 trap molecule is VGX-300. VGX-300 has the amino acid sequence set forth in SEQ ID NO: 7. In some embodiments, the soluble VEGFR-3 trap molecule comprises the amino acid sequence set forth in SEQ ID NO: 7. In some embodiments, the soluble VEGFR-3 trap molecule has an amino acid sequence consisting of the amino acid sequence set forth in SEQ ID NO: 7. In some embodiments, the soluble VEGFR-3 trap molecule has an amino acid sequence defined by positions 1-546 of SEQ ID NO: 7.

If needed, soluble VEGFR-3 trap molecules described herein may comprise a functional region facilitating purification or production. Specific examples of such additional amino acid sequences include a GST sequence or a His tag sequence. In some variations, the region facilitating purification is removed for formulation of a composition for pharmaceutical use.

Soluble VEGFR-3 trap molecules can be chemically modified (e.g., glycosylation, PEGylation, etc.) to impart desired characteristics. Such modifications preferably do not substantially reduce the growth factor binding affinities or specificities of the ligand binding molecule.

Polypeptides can be modified, for instance, by glycosylation, amidation, carboxylation, or phosphorylation, or by the creation of acid addition salts, amides, esters, in particular C-terminal esters, and N-acyl derivatives.

In some embodiments, a soluble VEGFR-3 trap molecule described herein optionally comprises at least one PEG (polyethylene glycol) moiety attached to the molecule. For example, in some embodiments, PEG of about 20-40 kDa is attached to the amino terminus of the ligand binding molecule. As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents. PEG is effective at excluding other polymers or peptides when present in water, primarily through its high dynamic chain mobility and hydrophilic nature, thus creating a water shell or hydration sphere when attached to other proteins or polymer surfaces. PEG is nontoxic, non-immunogenic, and approved by the Food and Drug Administration for internal consumption.

Polypeptides can be conjugated to a reporter group, including, but not limited to a radiolabel, a fluorescent label, an enzyme (e.g., that catalyzes a calorimetric or fluorometric reaction), a substrate, a solid matrix, or a carrier (e.g., biotin or avidin). Examples of analogs are described in WO 98/28621 and in Olofsson, et al., Proc. Nat'l. Acad. Sci. USA, 95:1 709-1 714 (1998), U.S. Pat. Nos. 5,512,545, and 5,474,982; U.S. patents application Ser. Nos. 20020164687 and 20020164710, the entire contents of each of which are incorporated herein by reference.

In some variations, the ligand binding molecule comprises a signal peptide that directs secretion of the molecule from a cell that expresses the molecule.

The soluble VEGFR-3 trap molecule may be prepared by any suitable process. For example, a cell line (e.g. a eukaryotic cell line, Chines Hamster Ovary cell line) may be transfected with a vector comprising a polynucleotide sequence encoding the amino acid sequence of the soluble VEGFR-3 trap molecule, and which is cultured to express the trap molecule. Methods for producing and purifying soluble VEGFR-3 trap molecules are described in WO2014/124487 A1 WO2015/123715 A1 and WO2002/060950 A1, the entire contents of each of which are incorporated herein by reference.

The soluble VEGFR-3 trap molecule is present in the aqueous pharmaceutical composition at a concentration in the range of from 5 mg/mL to 250 mg/mL.

In some embodiments, the soluble VEGFR-3 trap molecule is present at a concentration of from 10 mg/mL, of from 20 mg/mL, of from 30 mg/mL, of from 40 mg/mL, of from 50 mg/mL, of from 60 mg/mL, of from 70 mg/mL, of from 80 mg/mL, of from 90 mg/mL, of from 100 mg/mL, of from 110 mg/mL, of from 120 mg/mL, of from 130 mg/mL, of from 140 mg/mL, of from 150 mg/mL, of from 160 mg/mL, of from 170 mg/mL, of from 180 mg/mL of from 190 mg/mL, of from 200 mg/mL, of from 210 mg/mL, of from 220 mg/mL, of from 230 mg/mL, or of from 240 mg/mL. In some embodiments, the soluble VEGFR-3 trap molecule is present at a concentration of up to 240 mg/mL, up to 230 mg/mL, up to 220 mg/mL, up to 210 mg/mL, up to 200 mg/mL, up to 190 mg/mL, up to 180 mg/mL, up to 170 mg/mL, up to 160 mg/mL, up to 150 mg/mL, up to 140 mg/mL, up to 130 mg/mL, up to 120 mg/mL. up to 110 mg/mL, up to 100 mg/mL, up to 90 mg/mL, up to 80 mg/mL, up to 70 mg/mL, up to 60 mg/mL, up to 50 mg/mL, up to 40 mg/mL, up to 30 mg/mL, up to 20 mg/mL, or up to 10 mg/mL. Preferably, the soluble VEGFR-3 trap molecule is present a concentration of up to 120 mg/mL, more preferably up to 100 mg/mL, still more preferably up to 80 mg/mL, yet more preferably up to 60 mg/mL, further more preferably up to 40 mg/mL.

In some embodiments, the soluble VEGFR3 trap molecule is present at a concentration of from 5 mg/mL up to 120 mg/mL, of from 5 mg/mL up to 100 mg/mL, of from 5 mg/mL up to 80 mg/mL, of from 5 mg/mL up to 60 mg/mL, of from 5 mg/mL up to 50 mg/mL, of from 5 mg/mL up to 40 mg/mL, of from 10 mg/mL up to 120 mg/mL, of from 10 mg/mL up to 100 mg/mL, of from 10 mg/mL up to 80 mg/mL, of from 10 mg/mL up to 60 mg/mL, of from 10 mg/mL up to 50 mg/mL, of from 10 mg/mL up to 40 mg/mL, of from 20 mg/mL up to 120 mg/mL, of from 20 mg/mL up to 100 mg/mL, of from 20 mg/mL up to 80 mg/mL, of from 20 mg/mL up to 60 mg/mL, of from 20 mg/mL up to 50 mg/mL, of from 20 mg/mL up to 40 mg/mL, of from 30 mg/mL up to 120 mg/mL, of from 30 mg/mL up to 100 mg/mL, of from 30 mg/mL up to 80 mg/mL, of from 30 mg/mL up to 60 mg/mL, of from 30 mg/mL up to 50 mg/mL, or of from 30 mg/mL up to 40 mg/mL.

In some embodiments, the soluble VEGFR-3 trap molecule is present at a concentration of about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, or about 120 mg/mL.

### Excipients

The aqueous pharmaceutical compositions of the present disclosure contain trehalose. Trehalose is a disaccharide consisting of two glucose units joined by a 1,1-glycosidic bond. Typically, the glucose units present in trehalose are α-glucose units. Trehalose also has the names α,α-trehalose, α-D-glucopyranosyl-(1→1)-α-D-glucopyranoside, α-D-glucopyranosyl-α-D-glucopyranoside, and D-(+)-trehalose. Trehalose has the CAS no. 6138-23-4. Trehalose has the chemical structure:

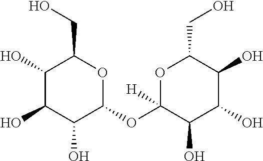 [SMILES: OC[C@H]1O[C@H](O[C@H]2O[C@H](CO)[C@@H](O)[C@H](O)[C@H]2O)[C@H](O)[C@@H](O)[C@@H]1O]

Forms of trehalose include the anhydrous and dihydrate forms. The molar mass of the anhydrous form of trehalose is 342.3 g/mol, and the molar mass of the dihydrate form of trehalose is 378.3 g/mol. Trehalose can be obtained from a variety of suppliers, for example Pfanstiel (www.pfanstiehl.com). Trehalose dihydrate is also available from Sigma Aldrich, Merck, Fisher Scientific, Acros, and Alfa Aesar.

In some embodiments, the aqueous pharmaceutical compositions of the present disclosure contain trehalose in a concentration of at least 7.0% w/v. Regardless of the form of trehalose used to produce the formulation (e.g. anhydrous trehalose, or trehalose dihydrate), the % w/v of trehalose relates to the percentage by weight of trehalose, and for example does not include any associated solvate (e.g. dihydrate). For example, a 10% w/v solution of trehalose may be prepared using 10 g of anhydrous trehalose and making up to 100 ml with water. Alternatively, a 10% w/v solution of trehalose may be prepared using 11.1 g of trehalose dihydrate and making up to 100 ml with water.

In some embodiments, trehalose is present at a concentration of up to 20% w/v, or up to 15% w/v, or up to 14% w/v, or up to 13% w/v, or up to 12% w/v, or up to 11% w/v. In some embodiments, trehalose is present at a concentration of at least 7.5% w/v, at least 8.0% w/v, at least 8.5% w/v, at least 9% w/v, at least 9.5% w/v, at least 10% w/v, or at least 10.5% w/v. In some embodiments, trehalose is present at a concentration of from 7.0% w/v to 20% w/v, or from 7.0% w/v to 15% w/v, or from 7.0% w/v to 14% w/v, or from 7.0% w/v to 13% w/v, or from 7.0% w/v to 12% w/v, or from 7.5% w/v to 20% w/v, or from 7.5% w/v to 15% w/v, or from 7.5% w/v to 14% w/v, or from 7.5% w/v to 13% w/v, or from 7.5% w/v to 12% w/v, or from 8.0% w/v to 20% w/v, or from 8.0% w/v to 15% w/v, or from 8.0% w/v to 14% w/v, or from 8.0% w/v to 13% w/v, or from 8.0% w/v to 12% w/v, or from 8.5% w/v to 20% w/v, or from 8.5% w/v to 15% w/v, or from 8.5% w/v to 14% w/v, or from 8.5% w/v to 13% w/v, or from 8.5% w/v to 12% w/v, or from 9% w/v to 20% w/v, or from 9% w/v to 15% w/v, or from 9% w/v to 14% w/v, or from 9% w/v to 13% w/v, or from 9% w/v to 12% w/v, or from 9.5% w/v to 20% w/v, or from 9.5% w/v to 15% w/v, or from 9.5% w/v to 14% w/v, or from 9.5% w/v to 13% w/v, or from 9.5% w/v to 12% w/v, or from 10% w/v to 20% w/v, or from 10% w/v to 15% w/v, or from 10% w/v to 14% w/v, or from 10% w/v to 13% w/v, or from 10% w/v to 12% w/v, or from 10.5% w/v to 20% w/v, or from 10.5% w/v to 15% w/v, or from 10.5% w/v to 14% w/v, or from 10.5% w/v to 13% w/v, or from 10.5% w/v to 12% w/v. In some embodiments, trehalose is present at a concentration of about 7.0% w/v, about 7.5% w/v, about 8.0% w/v, 8.5% w/v, about 9% w/v, about 9.5% w/v, about 10% w/v, about 10.1% w/v, about 10.2% w/v, about 10.3% w/v about 10.4% w/v, about 10.5% w/v, about 10.6% w/v, about 10.7% w/v, about 10.8% w/v, about 10.9% w/v, about 11% w/v, about 11.1% w/v, about 11.2% w/v, about 11.3% w/v, about 11.4% w/v, about 11.5% w/v, about 11.6% w/v, about 11.7% w/v, about 11.8% w/v, about 11.9% w/v, about 12% w/v, about 12.5% w/v, about 13% w/v, about 13.5% w/v, about 14% w/v, about 14.5% w/v, about 15% w/v, about 16% w/v, about 17% w/v, about 18% w/v, about 19% w/v, or about 20% w/v.

The aqueous pharmaceutical composition contains water. Typically sterile, high purity water is used, such as for example water for injection.

The aqueous pharmaceutical composition contains a buffer, e.g. for maintaining the pH in the desired range. Any suitable buffer may be utilised. Examples of buffers include a phosphate buffer (such as sodium dihydrogen phosphate, disodium phosphate), an amino acid such as a histidine buffer (e.g. histidine hydrochloride), a citrate buffer (e.g. sodium citrate), tris (2-Amino-2-(hydroxymethyl) propane-1,3-diol), and an acetate buffer (e.g. sodium acetate). In some embodiments, the buffer is tris or a phosphate buffer. In some embodiments, the buffer is a phosphate buffer, for example it may be a mixture of acidic and basic forms of phosphate. In some embodiments, the buffer is a sodium phosphate, for example a mixture of sodium dihydrogen phosphate and disodium phosphate.

The buffer is included at a suitable concentration, for example it may be present at a concentration of up to 100 mM, up to 90 mM, up to 80 mM, up to 70 mM, up to 60 mM, up to 50 mM, up to 40 mM, up to 30 mM, up to 20 mM, or up to 10 mM. In some embodiments, the buffer is present at a concentration of at least 5 mM, or at least 10 mM. In some embodiments, the buffer is present at a concentration in the range of from 5 mM to 100 mM, of from 5 mM to 80 mM, of from 5 mM to 70 mM, of from 5 mM to 60 mM, of from 5 mM to 50 mM, of from 5 mM to 40 mM, of from 5 mM to 30 mM, or of from 5 mM to 20 mM. In some embodiments, the buffer is included at a concentration of about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM.

It has been found that aqueous pharmaceutical compositions comprising a soluble VEGFR-3 trap molecule having higher pH have improved stability with regard to dimer formation.

The aqueous pharmaceutical composition has a pH in the range of from 6.5 to 8.0. In some embodiments, the pH of the composition is in the range of from 6.5 to 7.0, from 7.0 to 7.5, from 7.5 to 8.0, from 7.2 to 7.8, from 7.3 to 7.7, or from 7.4 to 7.6. In some embodiments, the pH of the composition is about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments, the aqueous pharmaceutical composition comprises a surfactant. Where a surfactant is included, it may for example be an ionic surfactant (e.g. cationic, anionic or zwitterionic) or non-ionic (e.g. neutral) surfactant. Examples of surfactants include polyoxyethylene (20) sorbitan monolaurate (e.g. sold under the brand names polysorbate 20®, Tween 20®), polyoxyethylene (20) sorbitan monooleate (e.g. sold under the brand names polysorbate 80® and Tween 80®), polyethylene glycol, and a poloxamer (e.g. a copolymer of poly(propylene oxide) and poly(ethylene oxide)) such as that sold under the brand name Pluronic F68®). In some embodiments, the aqueous pharmaceutical composition comprises a surfactant which is polyoxyethylene (20) sorbitan monolaurate or polyoxyethylene (20) sorbitan monooleate.

Where a surfactant is included, it may be present in a concentration in the range, for example, of from 0.005% to 0.2% w/v. In some embodiments, a surfactant is present at a concentration of from 0.005% to 0.1% w/v, or from 0.005% to 0.05% w/v, or from 0.005% to 0.02% w/v. In some embodiments, a surfactant is present at a concentration of about 0.005% w/v, or about 0.006% w/v, or about 0.007% w/v, or about 0.008% w/v, or about 0.009% w/v, or about 0.01% w/v, or about 0.011% w/v, or about 0.012% w/v, or about 0.013% w/v, or about 0.014% w/v, or about 0.015% w/v, or about 0.016% w/v, or about 0.017% w/v, or about 0.018% w/v or about 0.019% w/v, or about 0.02% w/v.

In some other embodiments, the aqueous pharmaceutical composition does not comprise a surfactant.

It has been found that aqueous pharmaceutical compositions containing a soluble VEGFR-3 trap molecule, and having relatively high osmolality, provide good stability properties. Osmolality relates to the concentration of osmotically active particles in solution, and is typically defined in units of mOsm/kg.

Accordingly, in some embodiments, the aqueous pharmaceutical composition has an osmolality of at least 300 mOsm/kg, or at least 350 mOsm/kg, or at least 400 mOsm/kg. In some embodiments, the aqueous pharmaceutical composition has an osmolality of up to 1000 mOsm/kg, or up to 900 mOsm/kg, or up to 800 mOsm/kg, or up to 700 mOsm/kg, or up to 600 mOsm/kg, or up to 500 mOsm/kg. In some embodiments, the aqueous pharmaceutical composition has an osmolality in the range of from 300 mOsm/kg to 1000 mOsm/kg, or from 350 mOsm/kg to 1000 mOsm/kg, or from 400 mOsm/kg to 1000 mOsm/kg, or from 300 mOsm/kg to 800 mOsm/kg, or from 350 mOsm/kg to 800 mOsm/kg, or from 400 mOsm/kg to 800 mOsm/kg, or from 300 mOsm/kg to 600 mOsm/kg, or from 350 mOsm/kg to 600 mOsm/kg, or from 400 mOsm/kg to 600 mOsm/kg.

The aqueous pharmaceutical compositions can contain relatively few components, but still provides good stability properties.

As defined herein, a tonicity agent is a substance which affects the osmolality of the pharmaceutical composition. Tonicity agents are typically included to adjust the osmolality of a composition to a desired value.

Whilst in some embodiments, the aqueous pharmaceutical composition comprises an additional tonicity agent, in other embodiments, the aqueous pharmaceutical composition does not contain an additional tonicity agent. The term additional tonicity agent refers to a substance which materially affects the osmolality of the pharmaceutical composition and which is other than the active agent, trehalose, buffer, water, and surfactant (if present). Examples of tonicity agents include sugars (e.g. sucrose, dextrose), certain salts (e.g. sodium chloride, potassium chloride), polyols (e.g. mannitol, sorbitol, glycerin).

In some embodiments, the pharmaceutical composition contains less than 50 mM sodium chloride, or less than 25 mM sodium chloride, or less than 10 mM sodium chloride, or less than 5 mM sodium chloride

In some embodiments, the pharmaceutical composition does not contain added sodium chloride. As used herein the term ‘does not contain added sodium chloride’ means that no sodium chloride is added during preparation of the pharmaceutical composition. It will be understood that the pharmaceutical composition may nevertheless include minor amounts of sodium chloride, for example if the pH of the formulation is adjusted by addition of hydrochloric acid and sodium hydroxide, some small quantity of sodium chloride may form.

In some embodiments, the aqueous pharmaceutical composition is substantially free of sodium chloride. In some embodiments, the composition contains less than 1 mM sodium chloride, or less than 0.5 mM sodium chloride, or less than 0.2 mM sodium chloride, or less than 0.1 mM sodium chloride, or less than 0.05 mM sodium chloride. In some embodiments, the aqueous pharmaceutical composition contains no detectable sodium chloride.

In some embodiments, the aqueous pharmaceutical composition is substantially free of additional sugar (i.e. it is substantially free of sugars (e.g. monosaccharides or disaccharides) other than trehalose and any sugars forming part of the active ingredient). In some embodiments, the aqueous pharmaceutical composition contains less than 1 mM additional sugar, less than 0.5 mM additional sugar, less than 0.2 mM additional sugar, less than 0.1 mM additional sugar, or less than 0.05 mM additional sugar. In some embodiments, the aqueous pharmaceutical composition contains no detectable additional sugar.

In some embodiments, the aqueous pharmaceutical composition does not contain any constituents beyond water, soluble VEGFR-3 trap molecule, a phosphate buffer, and surfactant (other than impurities which may be present in those constituents).

In some embodiments, the aqueous pharmaceutical composition comprises an active agent which is a soluble VEGFR-3 trap molecule, the active agent being present at a concentration in the range of from 5 mg/mL to 120 mg/mL,
trehalose in a concentration in the range of from 8.5% w/v to 20% w/v;
a buffer at a concentration in the range of from 5 mM to 20 mM;
optional surfactant; and
water;
wherein the pH of the aqueous pharmaceutical composition is in the range of from 6.5 to 8.0.

In some embodiments, the aqueous pharmaceutical composition comprises an active agent which is a soluble VEGFR-3 trap molecule, the active agent being present at a concentration in the range of from 20 mg/mL to 80 mg/mL,
trehalose in a concentration in the range of from 9% w/v to 13% w/v;
a buffer at a concentration in the range of from 5 mM to 20 mM, wherein the buffer is a sodium phosphate;
a surfactant which is polyoxyethylene (20) sorbitan monolaurate at a concentration of about 0.01% w/v; and
water;
wherein the pH of the aqueous pharmaceutical composition is in the range of from 7.0 to 8.0.

In some embodiments, the aqueous pharmaceutical composition consists or consists essentially of active agent, trehalose, buffer and surfactant.

In some embodiments, the aqueous pharmaceutical composition consists or consists essentially of active agent, trehalose, a sodium phosphate buffer, and polyoxyethylene (20) sorbitan monolaurate.

In some embodiments, the aqueous pharmaceutical composition consists or consists essentially of:
an active agent in a concentration of about 40 mg/ml, which is a soluble VEGFR-3 trap molecule that comprises a ligand binding polypeptide fused to an immunoglobulin constant domain fragment, the ligand binding polypeptide comprising immunoglobulin-like domains 1-3 of the extracellular domain of human VEGFR-3 and optionally having one or more modifications in an N-glycan region of the extracellular domain;
trehalose in a concentration of about 10.9% w/v;
a buffer in a concentration of about 10 mM, wherein the buffer is a sodium phosphate;
polyoxyethylene (20) sorbitan monolaurate in a concentration of about 0.01% w/v; and
   water;
wherein the pH of the aqueous pharmaceutical composition is about 7.5.

In some embodiments, the aqueous pharmaceutical composition consists or consists essentially of:
an active agent in a concentration of about 40 mg/ml, which is OPT-302 or VGX-300;
   trehalose in a concentration of about 10.9% w/v;
   a buffer in a concentration of about 10 mM, wherein the buffer is a sodium phosphate;
polyoxyethylene (20) sorbitan monolaurate in a concentration of about 0.01% w/v; and
   water;
wherein the pH of the aqueous pharmaceutical composition is about 7.5.

In some embodiments, the aqueous pharmaceutical composition consists or consists essentially of:
an active agent in a concentration of about 40 mg/ml, which comprises or consists of the amino acid sequence of any one of SEQ ID NOs.: 3, 4, 5 and 6;
   trehalose in a concentration of about 10.9% w/v;
   a buffer in a concentration of about 10 mM, wherein the buffer is a sodium phosphate;
polyoxyethylene (20) sorbitan monolaurate in a concentration of about 0.01% w/v; and
   water;
wherein the pH of the aqueous pharmaceutical composition is about 7.5.

In some embodiments, the aqueous pharmaceutical composition consists or consists essentially of:
an active agent in a concentration of about 40 mg/ml, which comprises or consists of the amino acid sequence of SEQ ID NO: 7;
trehalose in a concentration of about 10.9% w/v;
a buffer in a concentration of about 10 mM, wherein the buffer is a sodium phosphate;
polyoxyethylene (20) sorbitan monolaurate in a concentration of about 0.01% w/v; and
water;
wherein the pH of the aqueous pharmaceutical composition is about 7.5.

The aqueous pharmaceutical compositions of the may be prepared in advance and provided to hospitals, surgeries and the like as a pre-prepared aqueous pharmaceutical formulation. Alternatively, they may be provided as a solid composition, e.g. a lyophilised pharmaceutical composition, which is for reconstitution with water prior to use.

Accordingly, there is also provided a lyophilised pharmaceutical composition for reconstitution, comprising:
an active agent which is a soluble VEGFR-3 trap molecule;
trehalose; and
a buffer;
wherein the weight ratio of trehalose to active agent is in the range of from 1:3 to 40:1.

There is also provided a reconstituted pharmaceutical composition, wherein the pharmaceutical composition is obtained by admixing a lyophilised pharmaceutical composition as defined herein with an aqueous diluent.

In use, an appropriate quantity of aqueous diluent is added to the lyophilised pharmaceutical composition to achieve the desired concentration of the constituents (e.g. the desired concentration of active agent, trehalose and/or buffer).

A lyophilised pharmaceutical composition containing trehalose and active agent in a weight ratio range of from 1:3 to 40:1 corresponds to, following reconstitution with an appropriate quantity of aqueous diluent, a reconstituted pharmaceutical composition containing 5-250 mg/mL active agent, and 8.5% w/v to 20% w/v trehalose.

In some embodiments, the weight ratio of trehalose to active agent in the lyophilised pharmaceutical composition is in the range of from 1:1 to 7.5:1, from 1:1 to 5:1, or from 2.1:1 to 4.5:1.

In some embodiments, the weight ratio of trehalose to active agent in the lyophilised pharmaceutical composition is in the range of from 0.7:1 to 3.3:1, or from 0.8:1 to 3:1.

In some embodiments, the weight ratio of trehalose to active agent in the lyophilised pharmaceutical composition is about 2.7:1, or about 1.4:1, or about 0.9:1.

As described above, trehalose is a disaccharide consisting of two glucose units joined by a 1,1-glycosidic bond. Typically, the glucose units present in trehalose are α-glucose units. Trehalose also has the names α,α-trehalose, α-D-glucopyranosyl-(1→1)-α-D-glucopyranoside, α-D-glucopyranosyl-α-D-glucopyranoside, and D-(+)-trehalose. Trehalose has the CAS no. 6138-23-4. Trehalose has the chemical structure:

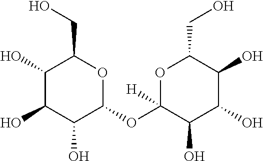 [SMILES: OC[C@H]1O[C@H](O[C@H]2O[C@H](CO)[C@@H](O)[C@H](O)[C@H]2O)[C@H](O)[C@@H](O)[C@@H]1O]

Forms of trehalose include the anhydrous and dihydrate forms. The molar mass of the anhydrous form of trehalose is 342.3 g/mol, and the molar mass of the dihydrate form of trehalose is 378.3 g/mol.

Trehalose can be obtained from a variety of suppliers, for example Pfanstiel (www.pfanstiehl.com). Trehalose dihydrate is also available from Sigma Aldrich, Merck, Fisher Scientific, Acros, and Alfa Aesar.

The lyophilised pharmaceutical composition contains a buffer. Any suitable buffer may be utilised. Examples of buffers include a phosphate buffer (such as sodium dihydrogen phosphate, disodium phosphate), an amino acid such as a histidine buffer (e.g. histidine hydrochloride), a citrate buffer (e.g. sodium citrate), tris (2-Amino-2-(hydroxymethyl) propane-1,3-diol), and an acetate buffer (e.g. sodium acetate). In some embodiments, the buffer is tris or a phosphate buffer. In some embodiments, the buffer is a phosphate buffer, for example it may be a mixture of acidic and basic forms of phosphate. In some embodiments, the buffer is a sodium phosphate, for example a mixture of sodium dihydrogen phosphate and disodium phosphate.

In some embodiments, the weight range of buffer to active agent is in the range of from 1:420 to 3:1, or from 1:200 to 1:1.8, or from 1:26 to 1:40.

In some embodiments, the buffer is a sodium phosphate, and the weight ratio of sodium phosphate to active agent is in the range of from 1:3 to 1:1000, or from 1:3 to 1:200, or from 1:5 to 1:100.

In some embodiments, the buffer is a sodium phosphate, and the weight ratio of sodium phosphate to active agent is about 0.03:1.

In some embodiments, the lyophilised pharmaceutical composition comprises a surfactant. Where a surfactant is included, it may for example be an ionic surfactant (e.g. cationic, anionic or zwitterionic) or non-ionic (e.g. neutral) surfactant. Examples of surfactants include polyoxyethylene (20) sorbitan monolaurate (e.g. sold under the brand names polysorbate 20®, Tween 20®), polyoxyethylene (20) sorbitan monooleate (e.g. sold under the brand names polysorbate 80® and Tween 80®), polyethylene glycol, and a poloxamer (e.g. a copolymer of poly(propylene oxide) and poly(ethylene oxide)) such as that sold under the brand name Pluronic F68®). In some embodiments, the lyophilised pharmaceutical composition comprises a surfactant which is polyoxyethylene (20) sorbitan monolaurate or polyoxyethylene (20) sorbitan monooleate.

The process of lyophilisation typically involves multiple steps, for example a freezing step, and one or more drying steps (e.g. a primary drying step involving sublimation, and a secondary drying step involving desorption).

The lyophilised pharmaceutical composition may for example contain one or more lyophilisation excipients. Examples of lyophilisation excipients include cryoprotectants an lyoprotectants. Examples of lyophilisation excipients include sugars such as sucrose, mannitol and dextrose, polymeric excipients such as polyvinylpyrrolidone, and amino acids such as glycine.

In some embodiments, the lyophilised pharmaceutical composition does not include any additional lyophilisation excipients (i.e. other than the active agent, trehalose, buffer, and surfactant, if present).

As discussed above, in use an appropriate quantity of aqueous diluent is added to the lyophilised pharmaceutical composition to achieve the desired concentration of the constituents (e.g. the desired concentration of active agent, trehalose and/or buffer). The aqueous diluent may for example be water. Typically sterile, high purity water is used, such as for example water for injection.

A quantity of aqueous diluent may be added such that the concentration of active agent in the reconstituted pharmaceutical composition is typically in the range of from 5 mg/mL to 250 mg/mL, or for example from 20 mg/mL to 120 mg/mL, or about 40 mg/mL, or about 80 mg/mL, or about 120 mg/mL.

As a further example, a quantity of aqueous diluent may be added such that the concentration of trehalose in the reconstituted pharmaceutical composition is typically at least 8.5% w/v, for example in the range of from 8.5% w/v to 20% w/v, or from 8.5% w/v to 15% w/v, or from 9% w/v to 13% w/v, or from 10% w/v to 12% w/v, or about 10.9% w/v.

As a still further example a quantity of aqueous diluent may be added such that the concentration of buffer is typically up to 100 mM, for example up to 50 mM, or up to 20 mM, or at least 5 mM, or in the range of from 5 mM to 50 mM, or in the range of from 5 mM to 20 mM, or about 5 mM, about 10 mM, about 15 mM, about 20 mM, about 25 mM, about 30 mM, about 35 mM, about 40 mM, about 45 mM, or about 50 mM.

Typically, following reconstitution, the pH of the reconstituted lyophilised pharmaceutical composition is in the range of from 6.5 to 8.0, for example in the range of from 6.5 to 7.0, from 7.0 to 7.5, from 7.5 to 8.0, from 7.2 to 7.8, from 7.3 to 7.7, or from 7.4 to 7.6. In some embodiments, the pH of the reconstituted lyophilised pharmaceutical composition is about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

The discussion above in relation to the aqueous pharmaceutical composition and the nature and amount of the required and optional features, such as the active agent, trehalose, buffer, surfactant, pH, osmolality, and tonicity agent, also applies to the lyophilised pharmaceutical composition and the reconstituted lyophilised pharmaceutical composition as appropriate.

### Composition Preparation

The pharmaceutical compositions of the present disclosure may be prepared by any suitable method. For example, an aqueous solution of purified soluble VEGFR-3 trap molecule may be subjected to buffer exchange processing steps, filtration, and/or admixing with other excipients as required.

In some embodiments, aqueous pharmaceutical compositions according to the present disclosure may be prepared by admixing an aqueous solution of soluble VEGFR-3 trap molecule with an aqueous trehalose solution, followed by subjecting the resulting mixture to ultrafiltration-diafiltration with buffer containing trehalose and sodium phosphate, followed by admixing with surfactant (e.g. polyoxyethylene (20) monolaurate).

In the case of lyophilised formulations, an aqueous pharmaceutical composition as described above may be prepared, and then subjected to lyophilisation. The aqueous composition may for example be subjected to low temperature conditions such that the mixture freezes, and subjected to low pressure conditions such that water is removed by sublimation.

### Composition Properties

Soluble VEGFR-3 trap molecules have poor stability properties and tend to form dimers or other high molecular weight aggregates on storage in aqueous pharmaceutical compositions, resulting in loss of purity, activity and reduced shelf life, or otherwise a need for low temperature storage conditions.

However, as demonstrated by the examples below, the aqueous pharmaceutical compositions of the present disclosure have demonstrated unexpectedly improved stability properties, with reduced dimer formation, and maintaining good levels of binding activity for VEGF-C and VEGF-D over time.

In some embodiments, the pharmaceutical composition forms less than 6%, or less than 5%, or less than 4% dimerised active agent following storage at 25° C. for a period of 2 months.

In some embodiments, the pharmaceutical composition forms less than 10%, or less than 8%, or less than 6%, or less than 5% dimerised active agent, following storage at 25° C. for a period of 3 months.

In some embodiments, the pharmaceutical composition forms less than less than 5%, or less than 4%, or less than 3%, or less than 2% dimerised active agent, following storage at 5° C. for a period of 2 months.

In some embodiments, the pharmaceutical composition forms less than 6%, or less than 5%, or less than 4%, or less than 3%, or less than 2% dimerised active agent, following storage at 5° C. for a period of 3 months.

In some embodiments, the pharmaceutical composition forms less than 6%, or less than 5%, or less than 4%, or less than 3%, or less than 2% dimerised active agent, following storage at 5° C. for a period of 6 months.

In some embodiments, the pharmaceutical composition forms less than 8%, or less than 6%, or less than 5%, or less than 4%, or less than 3%, or less than 2% dimerised active agent, following storage at 5° C. for a period of 12 months.

In some embodiments, the pharmaceutical composition forms less than 8%, or less than 6%, or less than 5%, or less than 4%, or less than 3%, or less than 2% dimerised active agent, following storage at 5° C. for a period of 18 months.

In some embodiments, the pharmaceutical composition forms less than 10%, or less than 8%, or less than 6%, or less than 5%, or less than 4%, or less than 3%, or less than 2% dimerised active agent, following storage at 5° C. for a period of 24 months.

In some embodiments, the pharmaceutical composition forms less than 10%, or less than 8%, or less than 6%, or less than 5%, or less than 4%, dimerised active agent, following storage at 5° C. for a period of 30 months.

In some embodiments, the pharmaceutical composition forms less than 5%, or less than 4%, or less than 3%, or less than 2% dimerised active agent, following storage at −20° C. for a period of 2 months.

In some embodiments, the pharmaceutical composition forms less than 5%, or less than 4%, or less than 3%, or less than 2% dimerised active agent, following storage at −20° C. for a period of 3 months.

In some embodiments, the pharmaceutical composition forms less than 10%, or less than 8%, or less than 6%, or less than 5%, or less than 4%, or less than 3%, or less than 2% dimerised active agent, following storage at −20° C. for a period of 6 months.

In some embodiments, the pharmaceutical composition forms less than 10%, or less than 8%, or less than 6%, or less than 5%, or less than 4%, or less than 3%, or less than 2% dimerised active agent, following storage at −20° C. for a period of 12 months.

In some embodiments, the pharmaceutical composition forms less than 10%, or less than 8%, or less than 6%, or less than 5%, or less than 4%, or less than 3%, or less than 2% dimerised active agent, following storage at −20° C. for a period of 18 months.

In some embodiments, the pharmaceutical composition forms less than 10%, or less than 8%, or less than 6%, or less than 5%, or less than 4%, or less than 3%, or less than 2% dimerised active agent, following storage at −20° C. for a period of 24 months.

In some embodiments, the pharmaceutical composition forms less than 10%, or less than 8%, or less than 6%, or less than 5%, or less than 4%, or less than 3%, or less than 2% dimerised active agent, following storage at −20° C. for a period of 30 months.

In some embodiments, the pharmaceutical composition retains at least 70%, or at least 80%, or at least 90% binding activity against VEGF-C and/or VEGF-D following storage at 5° C. for a period of 2 months, compared with the binding activity of the composition at 0 months.

In some embodiments, the pharmaceutical composition retains at least 70%, or at least 80%, or at least 90% binding activity against VEGF-C and/or VEGF-D following storage at 5° C. for a period of 3 months, compared with the binding activity of the composition at 0 months.

In some embodiments, the pharmaceutical composition retains at least 70%, or at least 80%, or at least 90% binding activity against VEGF-C and/or VEGF-D following storage at 5° C. for a period of 6 months, compared with the binding activity of the composition at 0 months.

In some embodiments, the pharmaceutical composition retains at least 80% binding activity against VEGF-C and/or VEGF-D following storage at 5° C. for a period of 12 months, compared with the binding activity of the composition at 0 months.

In some embodiments, the pharmaceutical composition retains at least 70% binding activity against VEGF-C and/or VEGF-D following storage at 5° C. for a period of 18 months, compared with the binding activity of the composition at 0 months.

In some embodiments, the pharmaceutical composition retains at least 60% binding activity against VEGF-C and/or VEGF-D following storage at 5° C. for a period of 24 months, compared with the binding activity of the composition at 0 months.

In some embodiments, the pharmaceutical composition retains at least 60% binding activity against VEGF-C and/or VEGF-D following storage at 5° C. for a period of 30 months, compared with the binding activity of the composition at 0 months.

In some embodiments, the pharmaceutical composition retains at least 70%, or at least 80%, or at least 90% binding activity against VEGF-C and/or VEGF-D following storage at −20° C. for a period of 2 months, compared with the binding activity of the composition at 0 months.

In some embodiments, the pharmaceutical composition retains at least 70%, or at least 80%, or at least 90% binding activity against VEGF-C and/or VEGF-D following storage at −20° C. for a period of 3 months, compared with the binding activity of the composition at 0 months.

In some embodiments, the pharmaceutical composition retains at least 70%, or at least 80%, or at least 90% binding activity against VEGF-C and/or VEGF-D following storage at −20° C. for a period of 6 months, compared with the binding activity of the composition at 0 months.

In some embodiments, the pharmaceutical composition retains at least 80% binding activity against VEGF-C and/or VEGF-D following storage at −20° C. for a period of 12 months, compared with the binding activity of the composition at 0 months.

In some embodiments, the pharmaceutical composition retains at least 80% binding activity against VEGF-C and/or VEGF-D following storage at −20° C. for a period of 18months, compared with the binding activity of the composition at 0 months.

In some embodiments, the pharmaceutical composition retains at least 80% binding activity against VEGF-C and/or VEGF-D following storage at −20° C. for a period of 24months, compared with the binding activity of the composition at 0 months.

In some embodiments, the pharmaceutical composition retains at least 80% binding activity against VEGF-C and/or VEGF-D following storage at −20° C. for a period of 30 months, compared with the binding activity of the composition at 0 months.

As defined herein, the shelf-life of a pharmaceutical composition is the period of time in months during which, on storage, the extent of dimerised active agent is less than 10%, and the binding activity against VEGF-C and/or VEGF-D remains at least 70% of that achieved at 0 months.

In some embodiments, the pharmaceutical composition has at least a 2 month shelf life, or at least a 3 month shelf life, on storage at 25° C.

In some embodiments, the pharmaceutical composition has at least a 3 month shelf life, or at least a 6 month shelf life, or at least a 12 month shelf life, or at least an 18 month shelf life, or at least a 24 month shelf life, or at least a 30 month shelf life, on storage at 5° C.

In some embodiments, the pharmaceutical composition has at least a 3 month shelf life, or at least a 6 month shelf life, or at least a 12 month shelf life, or at least an 18 month shelf life, or at least a 24 month shelf life, or at least a 30 month shelf life, on storage at −20° C.

In some embodiments, the pharmaceutical composition remains physically stable, i.e. there is no significant phase separation or precipitation of solid material, for at least 3 months, on storage at 25° C.

In some embodiments, the pharmaceutical composition remains physically stable, i.e. there is no significant phase separation or precipitation of solid material, for at least 3 months, at least 6 months, or at least 12 months, or at least 18 months, or at least 24 months, or at least 30 months, on storage at 5° C.

In some embodiments, the pharmaceutical composition remains physically stable, i.e. there is no significant phase separation or precipitation of solid material, for at least 3 months, at least 6 months, or at least 12 months, or at least 18 months, or at least 24 months, or at least 30 months, on storage at −20° C.

### Therapeutic Uses and Methods

The pharmaceutical compositions of the present disclosure find use in the therapy of diseases and/or disorders for which inhibition of the interaction of VEGF-C and/or VEGF-D with the VEGFR-3 receptor provides a therapeutic response. For example, the pharmaceutical compositions of the present disclosure are useful in inhibiting neovascularisation, and find use in the therapy of diseases and/or disorders associated with aberrant neovascularisation, angiogenesis and/or lymphangiogenesis.

Neovascularisation is the formation of new blood vessels. Angiogenesis is the formation of new blood vessels from existing blood vessels, and plays a role in range of conditions including cancers, and ocular disorders such as age-related macular degeneration. Lymphangiogenesis is the formation of lymphatic vessels from pre-existing lymphatic vessels, and excessive lymphatic vessel formation has been associated with a range of conditions including edema, neoplasm metastasis, and lymphangiomatosis.

Accordingly, the present disclosure also provides a method of inhibiting neovascularisation in a subject, comprising administering to the subject an effective amount of a pharmaceutical composition as defined herein.

The present disclosure also provides a method of treating and/or preventing a disease or disorder associated with aberrant neovascularisation, angiogenesis and/or lymphangiogenesis in a subject, comprising administering to the subject an effective amount of a pharmaceutical composition as defined herein. Also provided is use of a soluble VEGFR-3 trap molecule for the manufacture of a pharmaceutical composition as defined herein, for the treatment and/or prevention of a disease or disorder associated with aberrant neovascularisation, angiogenesis and/or lymphangiogenesis. Also provided herein is a pharmaceutical composition as defined herein for use in the treatment and/or prevention of a disease or disorder associated with aberrant neovascularisation, angiogenesis and/or lymphangiogenesis.

In some embodiments, the disease or disorder is an ocular disease or disorder. In some embodiments, the ocular disease of disorder is selected from the group consisting of macular degeneration, diabetic retinopathy, macular edema, retinal vein occlusion and macular telangiectasia. In some embodiments, the ocular disease or disorder is wet age-related macular degeneration. In some embodiments, the ocular disease or disorder is diabetic macular edema.

The pharmaceutical composition comprising a soluble VEGFR-3 trap molecule may also find use in the therapy of other diseases and/or disorders associated with aberrant neovascularisation, angiogenesis and/or lymphangiogenesis. In some embodiments, the disease or disorder is a cancer, for example in colorectal cancer, lung cancer, breast cancer, glioblastoma, ovarian cancer, cervical cancer, and renal cancer.

### Administration

The pharmaceutical composition may be administered by any suitable route, e.g. which is compatible with the disease or disorder to be treated.

In some embodiments, for example when the disease or disorder is an ocular condition, the pharmaceutical composition may be administered intravitreally. In some embodiments, the pharmaceutical composition is administered by intravitreal injection. Intravitreal injection involves administration into the vitreous humor of an eye.

In some embodiments, the pharmaceutical composition is administered using an implant device which is implanted into an eye, and which permits controlled release of active agent into the vitreous of the eye.

In some embodiments, the pharmaceutical composition is administered using a port device which is implanted in an eye, which port device comprises a reservoir for the pharmaceutical composition, and permits controlled release of active agent into the vitreous of the eye.

In some embodiments, the pharmaceutical composition may be administered intravenously. In some embodiments, the pharmaceutical composition may be administered subcutaneously. In some embodiments, the pharmaceutical composition may be administered intramuscularly. In some embodiments, the pharmaceutical composition may be administered intrathecally.

In some embodiments, the pharmaceutical composition is administered by injection. In some embodiments, the pharmaceutical composition is administered by infusion.

The pharmaceutical composition may accordingly be formulated for injection (e.g. for intravitreal injection, subcutaneous objection, intravenous injection, intramuscular injection, or intrathecal injection), or for administration using an ocular implant device.

In use, a suitable dosage amount and dosing regime of the pharmaceutical composition comprising the soluble VEGFR-3 trap molecule is utilised. The amount and frequency of dosing may depend on factors including the type of disease or disorder, whether the active agent is being administered for prevention or treatment purpose, the age, weight, sex and health of the person to be treated, the route of administration, and whether the soluble VEGFR-3 trap molecule is administered in combination with other active agents. Additionally, pharmacogenomic information (the effect of genotype on the pharmacokinetic, pharmacodynamic or efficacy profile of a therapeutic) about a particular patient may affect dosage the used.

### Dosage Forms

The pharmaceutical compositions may be administered in a range of dosage forms. The present disclosure accordingly also includes a container containing the pharmaceutical composition. The present disclosure also includes a kit comprising a container containing the pharmaceutical composition, and optionally comprising instructions for administering the pharmaceutical composition to a subject.

For example, in the case of the aqueous pharmaceutical formulation, the pharmaceutical composition may be provided in a vial or bottle, and administered using a syringe. In a further example, the aqueous pharmaceutical composition may be provided in a pre-filled syringe. Accordingly, in some embodiments, the kit comprises a device for administering the pharmaceutical composition.

Where multiple doses are to be delivered (e.g. over time to the same subject, or to different subjects), for convenience a container containing multiple separate sections each containing a unit dosage of the pharmaceutical composition may be used for example (e.g. a divided bottle or container having multiple wells). As an alternative, a kit comprising multiple containers each containing a unit dosage of the pharmaceutical composition may be utilised.

As described above, in some embodiments the pharmaceutical composition may be administered utilising a port device which may be implanted in an eye. Accordingly, there is also provided a port device for implantation in an eye, the port device comprising a reservoir containing a pharmaceutical composition as defined herein, and wherein the port device permits controlled release of active agent into the vitreous of the eye.

In use, the port device may for example be implanted through the surface of the eye, e.g. through the sclera, such that it can release active agent into the vitreous, but at least a portion of the device can be accessible for re-filling of the reservoir.

In some embodiments, the port device may comprise a reservoir chamber coupled to a membrane, an opening, a diffusion barrier, a diffusion mechanism and/or porous structure for controlled release of the active agent. For example it may contain, e.g., a semipermeable membrane that permits passive diffusion of the active agent into the vitreous of the eye, such as a titanium-containing semipermeable membrane.

The port device will typically contain one or more retention elements for retaining the device in position, e.g. through the sclera. The port device may for example extend through the sclera but be covered by the conjunctiva.

The port device may be refillable, for example it may contain a re-filling element, such as a septum (e.g. a silicone septum) which permits refilling of the reservoir with additional pharmaceutical composition using a needle.

In some embodiments, the port device is configured to receive an amount of pharmaceutical composition sufficient to deliver a therapeutic dosage of active agent for up to 2 weeks, up to 3 weeks, up to 4 weeks, up to 1 month, up to 2 months, up to 3 months, up to 4 months, up to 5 months, or up to 6 months.

Examples of port devices are disclosed in WO2012/019176, WO2012/065006 and WO2014/152959, the contents of each of which are incorporated herein by reference in their entirety.

Also provided herein is a kit comprising (i) a port device for implantation in an eye, the port device comprising a reservoir for containing a pharmaceutical composition as defined herein, wherein the port device permits controlled release of active agent into the vitreous of the eye; and (ii) a container comprising an amount of pharmaceutical composition as defined herein, for filling of the port device. In some embodiments, the kit comprises a syringe for filling the port device with the pharmaceutical composition. In some embodiments, the container comprising the pharmaceutical composition is a syringe for filling of the port device.

As described above, in the case of a lyophilised pharmaceutical composition, in use the lyophilised pharmaceutical composition may be reconstituted by admixing with an aqueous diluent prior to administration. Accordingly, there is also provided a kit for reconstitution comprising a lyophilised pharmaceutical composition as defined herein, and an aqueous diluent. The aqueous diluent may for example be sterile water, e.g. water for injection.

### Combination Therapy

Whilst in some embodiments, the soluble VEGFR-3 trap molecule may be administered via the pharmaceutical compositions of the present disclosure as monotherapy, in other embodiments it is administered as part of a combination therapy treatment regime, e.g. in combination with a further active agent, for example a further active agent useful for the treatment and/or prevention of a disease or disorder associated with aberrant neovascularisation, angiogenesis and/or lymphangiogenesis.

The pharmaceutical composition comprising the soluble VEGFR-3 trap molecule may for example be administered with a further active agent simultaneously, sequentially, or separately. For example, a course of therapy may be prescribed for a patient involving administration of the soluble VEGFR-3 trap molecule at certain timepoints, and involving administration of one or more further active agents at different timepoints.

The further active agent may, for example, be a further active agent useful for the treatment and/or prevention of an ocular disease or disorder, e.g. an ocular disease or disorder selected from the group consisting of macular degeneration (e.g. wet age-related macular degeneration), diabetic retinopathy, macular edema (e.g. diabetic macular edema), retinal vein occlusion and macular telangiectasia.

In some embodiments, the further active agent is an anti-VEGF-A agent or an ant-VEGF-B agent. Examples of anti-VEGF-A and/or anti-VEGF-B agents include ranibizumab (Lucentis®), aflibercept (Eylea®), bevacizumab (Avastin®) and brolucizumab (Beovu®).

In some embodiments, the further active agent is pegaptanib (Macugen®).

In some embodiments the further active agent is a steroid, for example triamcinolone acetonide, dexamethasone (Ozurdex®), or fluocinolone acetonide (Retisert®, Iluvien®).

In some embodiments, the pharmaceutical composition comprising the soluble VEGFR-3 trap molecule is administered in combination with photodynamic therapy. Photodynamic therapy involves administration of a photosensitive active agent (e.g. verteporfin (Visudyne®) in conjunction with laser treatment. In some embodiments, the pharmaceutical composition comprising the soluble VEGFR-3 trap molecule is administered in combination with laser photocoagulation therapy. Laser photocoagulation therapy involves direction of a concentrated beam of high energy laser light directed on to the retina, to seal leaky blood vessels.

In some embodiments, the pharmaceutical composition comprising the soluble VEGFR-3 trap molecule is administered in combination with focal-grid macular laser surgery.

The pharmaceutical composition of the present disclosure may, for example, be packaged together with another pharmaceutical composition containing further active agent, e.g. in a pack or kit containing both medications.

All publications and patents mentioned herein are hereby incorporated by reference in their entirety as if each individual publication or patent was specifically and individually indicated to be incorporated by reference. In case of conflict, the present application, including any definitions herein, will control.

Sequence Listing

| | |
|---|---|
| Sequences | |
| Sequence Number [ID] | 1 |
| Molecule Type | AA |
| Length | 764 |
| | source 1 . . . 764 |
| Features Location/Qualifiers | mol_type = protein |
| | organism = Homo sapiens |
| NonEnglishQualifier Value | |
| Residues | |

MQRGAALCLR LWLCLGLLDG LVSGYSMTPP TLNITEESHV IDTGDSLSIS CRGQHPLEWA 60
WPGAQEAPAT GDKDSEDTGV VRDCEGTDAR PYCKVLLLHE VHANDTGSYV CYYKYIKARI 120
EGTTAASSYV FVRDFEQPFI NKPDTLLVNR KDAMWVPCLV SIPGLNVTLR SQSSVLWPDG 180
QEVVWDDRRG MLVSTPLLHD ALYLQCETTW GDQDFLSNPF LVHITGNELY DIQLLPRKSL 240
ELLVGEKLVL NCTVWAEFNS GVTFDWDYPG KQAERGKWVP ERRSQQTHTE LSSILTIHNV 300
SQHDLGSYVC KANNGIQRFR ESTEVIVHEN PFISVEWLKG PILEATAGDE LVKLPVKLAA 360
YPPPEFQWYK DGKALSGRHS PHALVLKEVT EASTGTYTLA LWNSAAGLRR NISLELVVNV 420
PPQIHEKEAS SPSIYSRHSR QALTCTAYGV PLPLSIQWHW RPWTPCKMFA QRSLRRRQQQ 480
DLMPQCRDWR AVTTQDAVNP IESLDTWTEF VEGKNKTVSK LVIQNANVSA MYKCVVSNKV 540
GQDERLIYFY VTTIPDGFTI ESKPSEELLE GQPVLLSCQA DSYKYEHLRW YRLNLSTLHD 600
AHGNPLLLDC KNVHLFATPL AASLEEVAPG ARHATLSLSI PRVAPEHEGH YVCEVQDRRS 660
HDKHCHKKYL SVQALEAPRL TQNLTDLLVN VSDSLEMQCL VAGAHAPSIV WYKDERLLEE 720
KSGVDLADSN QKLSIQRVRE EDAGRYLCSV CNAKGCVNSS ASVA 764

| | |
|---|---|
| Sequences | |
| Sequence Number [ID] | 2 |
| Molecule Type | AA |
| Length | 330 |
| | source 1 . . . 330 |
| Features Location/Qualifiers | mol_type = protein |
| | organism = Homo sapiens |
| NonEnglishQualifier Value | |

Residues
ASTKGPSVFP LAPSSKSTSG GTAALGCLVK DYFPEPVTVS WNSGALTSGV HTFPAVLQSS 60
GLYSLSSVVT VPSSSLGTQT YICNVNHKPS NTKVDKKVEP KSCDKTHTCP PCPAPELLGG 120
PSVFLFPPKP KDTLMISRTP EVTCVVVDVS HEDPEVKFNW YVDGVEVHNA KTKPREEQYN 180
STYRVVSVLT VLHQDWLNGK EYKCKVSNKA LPAPIEKTIS KAKGQPREPQ VYTLPPSRDE 240
LTKNQVSLTC LVKGFYPSDI AVEWESNGQP ENNYKTTPPV LDSDGSFFLY SKLTVDKSRW 300
QQGNVFSCSV MHEALHNHYT QKSLSLSPGK 330
Sequences

| | |
|---|---|
| Sequence Number [ID] | 3 |
| Molecule Type | AA |
| Length | 537 |
| | source 1 . . . 537 |
| Features Location/Qualifiers | mol_type = protein |
| | organism = synthetic construct |
| NonEnglishQualifier Value | |
| Features Location/Qualifiers | CARBOHYD 9 . . . 11 |
| | note = N-Glycosylation |
| NonEnglishQualifier Value | |
| Features Location/Qualifiers | CARBOHYD 142 . . . 144 |
| | note = N-Glycosylation |
| NonEnglishQualifier Value | |
| Features Location/Qualifiers | CARBOHYD 227 . . . 229 |
| | note = N-Glycosylation |
| NonEnglishQualifier Value | |
| Features Location/Qualifiers | CARBOHYD 275 . . . 277 |
| | note = N-Glycosylation |
| NonEnglishQualifier Value | |
| Features Location/Qualifiers | CARBOHYD 387 . . . 389 |
| | note = N-Glycosylation |
| NonEnglishQualifier Value | |
| Features Location/Qualifiers | DOMAIN 1 . . . 305 |
| | note = VEGFR-3 ECD 1-3 Portion |
| NonEnglishQualifier Value | |
| Features Location/Qualifiers | DOMAIN 306 . . . 537 |
| | note = Fc Portion |
| NonEnglishQualifier Value | |

Residues
YSMTPPTLNI TEESHVIDTG DSLSISCRGQ HPLEWAWPGA QEAPATGDKD SEDTGVVRDC 60
EGTDARPYCK VLLLHEVHAQ DTGSYVCYYK YIKARIEGTT AASSYVFVRD FEQPFINKPD 120
TLLVNRKDAM WVPCLVSIPG LNVTLRSQSS VLWPDGQEVV WDDRRGMLVS TPLLHDALYL 180
QCETTWGDQD FLSNPFLVHI TGNELYDIQL LPRKSLELLV GEKLVLNCTV WAEFNSGVTF 240
DWDYPGKQAE RGKWVPERRS QQTHTELSSI LTIHNVSQHD LGSYVCKANN GIQRFRESTE 300
VIVHEEPKSC DKTHTCPPCP APELLGGPSV FLFPPKPKDT LMISRTPEVT CVVVDVSHED 360
PEVKFNWYVD GVEVHNAKTK PREEQYNSTY RVVSVLTVLH QDWLNGKEYK CKVSNKALPA 420
PIEKTISKAK GQPREPQVYT LPPSRDELTK NQVSLTCLVK GFYPSDIAVE WESNGQPENN 480
YKTTPPVLDS DGSFFLYSKL TVDKSRWQQG NVFSCSVMHE ALHNHYTQKS LSLSPGK 537

| | |
|---|---|
| Sequence | |
| Sequence Number [ID] | 4 |
| Molecule Type | AA |
| Length | 537 |
| | source 1 . . . 537 |
| Features Location/Qualifiers | mol_type = protein |
| | organism = synthetic construct |
| NonEnglishQualifier Value | |
| Features Location/Qualifiers | CARBOHYD 9 . . . 11 |
| | note = N-Glycosylation |
| NonEnglishQualifier Value | |
| Features Location/Qualifiers | CARBOHYD 142 . . . 144 |
| | note = N-Glycosylation |
| NonEnglishQualifier Value | |
| Features Location/Qualifiers | CARBOHYD 227..229 |
| | note = N-Glycosylation |
| NonEnglishQualifier Value | |
| Features Location/Qualifiers | CARBOHYD 275 . . . 277 |
| | note = N-Glycosylation |
| NonEnglishQualifier Value | |
| Features Location/Qualifiers | CARBOHYD 387 . . . 389 |
| | note = N-Glycosylation |
| NonEnglishQualifier Value | |
| Features Location/Qualifiers | DOMAIN 1.305 |
| | note = VEGFR-3 ECD 1-3 Portion |
| NonEnglishQualifier Value | |
| Features Location/Qualifiers | DOMAIN 306 . . . 537 |
| | note = Fc Portion |
| | SITE 310 |
| | note = Substitution of C with S |
| NonEnglishQualifier Value | |

Residues
YSMTPPTLNI TEESHVIDTG DSLSISCRGQ HPLEWAWPGA QEAPATGDKD SEDTGVVRDC 60
EGTDARPYCK VLLLHEVHAQ DTGSYVCYYK YIKARIEGTT AASSYVFVRD FEQPFINKPD 120
TLLVNRKDAM WVPCLVSIPG LNVTLRSQSS VLWPDGQEVV WDDRRGMLVS TPLLHDALYL 180
QCETTWGDQD FLSNPFLVHI TGNELYDIQL LPRKSLELLV GEKLVLNCTV WAEFNSGVTF 240
DWDYPGKQAE RGKWVPERRS QQTHTELSSI LTIHNVSQHD LGSYVCKANN GIQRFRESTE 300
VIVHEEPKSS DKTHTCPPCP APELLGGPSV FLFPPKPKDT LMISRTPEVT CVVVDVSHED 360
PEVKFNWYVD GVEVHNAKTK PREEQYNSTY RVVSVLTVLH QDWLNGKEYK CKVSNKALPA 420
PIEKTISKAK GQPREPQVYT LPPSRDELTK NQVSLTCLVK GFYPSDIAVE WESNGQPENN 480
YKTTPPVLDS DGSFFLYSKL TVDKSRWQQG NVFSCSVMHE ALHNHYTQKS LSLSPGK 537
Sequences

| | |
|---|---|
| Sequence Number [ID] | 5 |
| Molecule Type | AA |
| Length | 547 |
| | source 1 . . . 547 |
| Features Location/Qualifiers | mol_type = protein |
| | organism = synthetic construct |
| | note = N-Glycosylation |
| NonEnglishQualifier Value | |
| Features Location/Qualifiers | CARBOHYD 227 . . . 229 |
| | note = N-Glycosylation |
| NonEnglishQualifier Value | |
| Features Location/Qualifiers | CARBOHYD 275 . . . 277 |
| | note = N-Glycosylation |
| NonEnglishQualifier Value | |
| Features Location/Qualifiers | CARBOHYD 397 . . . 399 |
| | note = N-Glycosylation |
| NonEnglishQualifier Value | |
| Features Location/Qualifiers | DOMAIN 1 . . . 305 |
| | note = VEGFR-3 ECD 1-3 Portion |
| NonEnglishQualifier Value | |
| Features Location/Qualifiers | DOMAIN 316 . . . 547 |
| | note = Fc Portion |
| NonEnglishQualifier Value | |

Residues
YSMTPPTLNI TEESHVIDTG DSLSISCRGQ HPLEWAWPGA QEAPATGDKD SEDTGVVRDC 60
EGTDARPYCK VLLLHEVHAQ DTGSYVCYYK YIKARIEGTT AASSYVFVRD FEQPFINKPD 120
TLLVNRKDAM WVPCLVSIPG LNVTLRSQSS VLWPDGQEVV WDDRRGMLVS TPLLHDALYL 180
QCETTWGDQD FLSNPFLVHI TGNELYDIQL LPRKSLELLV GEKLVLNCTV WAEFNSGVTF 240
DWDYPGKQAE RGKWVPERRS QQTHTELSSI LTIHNVSQHD LGSYVCKANN GIQRFRESTE 300
VIVHENPFIS VEWLKEPKSC DKTHTCPPCP APELLGGPSV FLFPPKPKDT LMISRTPEVT 360
CVVVDVSHED PEVKFNWYVD GVEVHNAKTK PREEQYNSTY RVVSVLTVLH QDWLNGKEYK 420
CKVSNKALPA PIEKTISKAK GQPREPQVYT LPPSRDELTK NQVSLTCLVK GFYPSDIAVE 480
WESNGQPENN YKTTPPVLDS DGSFFLYSKL TVDKSRWQQG NVFSCSVMHE ALHNHYTQKS 540
LSLSPGK 547

| | |
|---|---|
| Sequences | |
| Sequence Number [ID] | 6 |
| Molecule Type | AA |
| Length | 547 |
| | source 1 . . . 547 |
| Features Location/Qualifiers | mol_type = protein |
| | organism = synthetic construct |
| NonEnglishQualifier Value | |
| Features Location/Qualifiers | CARBOHYD 9 . . . 11 |
| | note = N-Glycosylation |
| NonEnglishQualifier Value | |
| Features Location/Qualifiers | CARBOHYD 142 . . . 144 |
| | note = N-Glycosylation |
| NonEnglishQualifier Value | |
| Features Location/Qualifiers | CARBOHYD 227 . . . 229 |
| | note = N-Glycosylation |
| NonEnglishQualifier Value | |
| Features Location/Qualifiers | CARBOHYD 275 . . . 277 |
| | note = N-Glycosylation |
| NonEnglishQualifier Value | |
| Features Location/Qualifiers | CARBOHYD 397 . . . 399 |
| | note = N-Glycosylation |
| NonEnglishQualifier Value | |
| Features Location/Qualifiers | DOMAIN 1 . . . 305 |
| | note = VEGFR-3 ECD 1-3 Portion |
| NonEnglishQualifier Value | |
| Features Location/Qualifiers | DOMAIN 316 . . . 547 |
| | note = Fc Portion |
| NonEnglishQualifier Value | |
| Features Location/Qualifiers | SITE 320 |
| | note = Substitution of C with S |
| NonEnglishQualifier Value | |

Residues
YSMTPPTLNI TEESHVIDTG DSLSISCRGQ HPLEWAWPGA QEAPATGDKD SEDTGVVRDC 60
EGTDARPYCK VLLLHEVHAQ DTGSYVCYYK YIKARIEGTT AASSYVFVRD FEQPFINKPD 120
TLLVNRKDAM WVPCLVSIPG LNVTLRSQSS VLWPDGQEVV WDDRRGMLVS TPLLHDALYL 180
TGNELYDIQL GEKLVLNCTV WAEFNSGVTF DWDYPGKQAE RGKWVPERRS LTIHNVSQHD 240
DWDYPGKQAE RGKWVPERRS TGNELYDIQL LPRKSLELLV GEKLVLNCTV WAEFNSGVTF 240
QCETTWGDQD FLSNPFLVHI QQTHTELSSI LTIHNVSQHD LGSYVCKANN GIQRFRESTE 300
VIVHENPFIS VEWLKEPKSS DKTHTCPPCP APELLGGPSV FLFPPKPKDT LMISRTPEVT 360
CVVVDVSHED PEVKFNWYVD GVEVHNAKTK PREEQYNSTY RVVSVLTVLH QDWLNGKEYK 420
CKVSNKALPA PIEKTISKAK GQPREPQVYT LPPSRDELTK NQVSLTCLVK GFYPSDIAVE 480
WESNGQPENN YKTTPPVLDS DGSFFLYSKL TVDKSRWQQG NVFSCSVMHE ALHNHYTQKS 540
LSLSPGK 547

| | |
|---|---|
| Sequences | |
| Sequence Number [ID] | 7 |
| Molecule Type | AA |
| Length | 548 |
| | source 1 . . . 548 |
| Features Location/Qualifiers | mol_type = protein |
| | organism = synthetic construct |
| NonEnglishQualifier Value | |
| Features Location/Qualifiers | CARBOHYD 9 . . . 11 |
| | note = N-Glycosylation |
| NonEnglishQualifier Value | |
| Features Location/Qualifiers | CARBOHYD 80 . . . 82 |
| | note = N-Glycosylation |
| NonEnglishQualifier Value | |
| Features Location/Qualifiers | CARBOHYD 142 . . . 144 |
| | note = N-Glycosylation |
| NonEnglishQualifier Value | |
| Features Location/Qualifiers | CARBOHYD 227 . . . 229 |
| | note = N-Glycosylation |
| NonEnglishQualifier Value | |
| Features Location/Qualifiers | CARBOHYD 275 . . . 277 |
| | note = N-Glycosylation |
| NonEnglishQualifier Value | |
| Features Location/Qualifiers | CARBOHYD 398 . . . 400 |
| | note = N-Glycosylation |
| NonEnglishQualifier Value | |
| Features Location/Qualifiers | DOMAIN 1 . . . 305 |
| | note = VEGFR-3 ECD 1-3 Portion |
| NonEnglishQualifier Value | |
| Features Location/Qualifiers | note = Factor Xa linker |
| | DOMAIN 306 . . . 316 |
| NonEnglishQualifier Value | |
| Features Location/Qualifiers | DOMAIN 317 . . . 548 |
| | note = Fc Portion |
| NonEnglishQualifier Value | |
| Residues | |

YSMTPPTLNI TEESHVIDTG DSLSISCRGQ HPLEWAWPGA QEAPATGDKD SEDTGVVRDC 60
EGTDARPYCK VLLLHEVHAN DTGSYVCYYK YIKARIEGTT AASSYVFVRD FEQPFINKPD 120
TLLVNRKDAM WVPCLVSIPG LNVTLRSQSS VLWPDGQEVV WDDRRGMLVS TPLLHDALYL 180
QCETTWGDQD FLSNPFLVHI TGNELYDIQL LPRKSLELLV GEKLVLNCTV WAEFNSGVTF 240
DWDYPGKQAE RGKWVPERRS QQTHTELSSI LTIHNVSQHD LGSYVCKANN GIQRFRESTE 300
VIVHEDPIEG RGGGGGDPKS CDKPHTCPLC PAPELLGGPS VFLFPPKPKD TLMISRTPEV 360
TCVVVDVSHE DPEVKFNWYV DGVEVHNAKT KPREEQYNST YRVVSVLTVL HQDWLNGKEY 420
KCKVSNKALP APIEKTISKA KGQPREPQVY TLPPSRDELT KNQVSLTCLV KGFYPSDIAV 480
EWESNGQPEN NYKATPPVLD SDGSFFLYSK LTVDKSRWQQ GNVFSCSVMH EALHNHYTQK 540
SLSLSPGK 548

| | |
|---|---|
| Sequences | |
| Sequence Number [ID] | 8 |
| Molecule Type | AA |
| Length | 11 |
| | source 1 . . . 11 |
| Features Location/Qualifiers | mol_type = protein |
| | organism = synthetic construct |
| NonEnglishQualifier Value | |
| Residues | |

DPIEGRGGGG G 11

### EXAMPLES

The present disclosure is further exemplified by the following non-limiting examples.

### Example 1: Initial Formulation Screening and DOE (Design of Experiments) Study

A formulation study was undertaken to identify preferred formulations. pH, buffers and excipients were screened and a Design of Experiments (DOE) was conducted.

Briefly, initial high-throughput screens were performed using one or more of Differential Scanning calorimetry/Fluorimetry (DSC/F) and Dynamic Light Scattering (DLS). Subsequent experiments focused on Size Exclusion Chromatography (SEC) and ELISA (VEGF-D) to assess formulation stability.

The results from pH/buffer screens showed an increase in thermal unfolding temperatures in buffers from pH 6.5 to 8.0, indicative of increased thermal stability.

Results from an initial excipient screen found that sodium and trehalose impart higher thermal stability in citrate, phosphate, and tris buffer.

A 1-week accelerated stability study, in the same buffers with a panel of excipients, found that tris and phosphate buffer with mannitol, trehalose and proline showed the least amount of degradation by SEC.

A surfactant study showed that OPT-302 when formulated in 20 mM tris buffer at pH 8.0 had the least amount of total aggregate and dimer formation, when trehalose and proline were excipients, and that the inclusion of PS-20 (polysorbate 20) into the formulations had minimal impact on stability.

The results of the DOE solution stability study found that the stability of the OPT-302 formulated in tris buffer increased with increasing pH and increasing concentration of trehalose.

From the in-design and off-design formulations from the DOE study, formulations were identified as possible candidates for additional study:
40 mg/mL OPT-302 in 20 mM tris, pH 8.0, 18.1% w/v trehalose, 0.01% w/v PS-20, with or without 100 mM proline;
40 mg/mL OPT-302 in 20 mM phosphate buffer at pH 7.5, 18.1% trehalose, and 0.01% w/v PS-20.

### Example 2: Effect of Trehalose Content and pH on OPT-302 Formulation Stability

Formulations of OPT-302 were prepared and their tendency to form dimers of active ingredient over time was analysed. The formulations contained 40 mg/mL OPT-302, water, 10 mM sodium phosphate buffer, and 0.01% polysorbate 20. The formulations were at pH 7.50 or 7.20, and contained either 9.0% w/v, 13.6% w/v or 18.1% w/v trehalose (prepared respectively using 10% w/v, 15% w/v and 20% w/v trehalose dihydrate).

Formulations were incubated for 1 week at 37° C., and analysed by SE-HPLC. The results showed a trend towards decreased dimerization with increasing trehalose content.

| Formulation (10 mM | | | |
|---|---|---|---|
| phosphate, 0.01% PS-20) | | % Dimer | |
| Trehalose | | | 1 week at |
| (% w/v) | pH | T = 0 | 37° C. |
| 9.0 | 7.50 | 3.8 | 4.3 |
| 13.6 | 7.50 | 3.4 | 4.0 |
| 18.1 | 7.50 | 4.1 | 2.9 |
| 9.0 | 7.20 | 4.3 | 6.5 |
| 13.6 | 7.20 | 3.8 | 5.1 |
| 18.1 | 7.20 | 4.4 | 4.4 |

### Example 3: Effect of Trehalose Content and Sodium Chloride Content on OPT-302 Formulation Stability

Formulations of OPT-302 were prepared and their tendency to form high molecular weight species (e.g. dimers) of active ingredient over time was analysed. The formulations contained 40 mg/mL OPT-302, water, and 10 mM sodium phosphate buffer, at pH 7.4. The formulations contained either 4.5% w/v, 6.8% w/v, 9.0% w/v or 18.1% w/v trehalose (prepared respectively using 5% w/v, 7.5% w/v, 10% w/v and 20% w/v trehalose dihydrate), and either 40 mM, 100 mM or 140 mM sodium chloride.

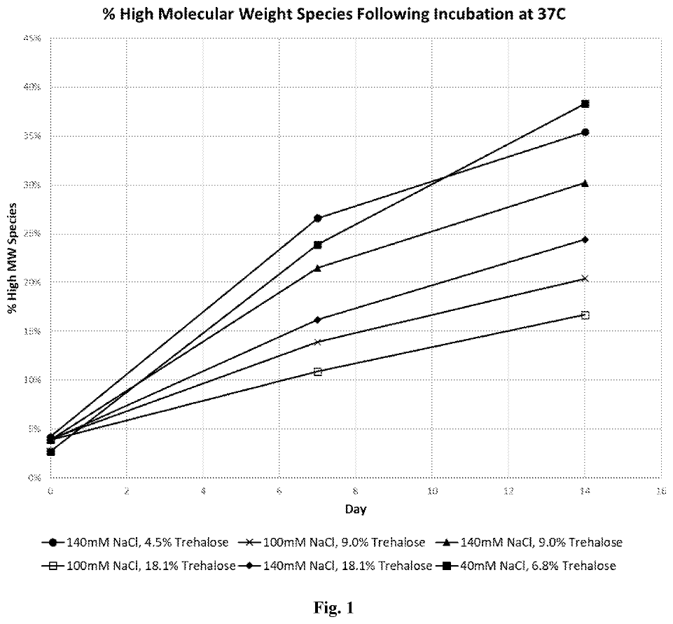

Formulations were incubated for up to 2 weeks at 37° C., and analysed by SE-HPLC. The results are shown in FIG. 1. The rate of dimerization formation was highest for formulations containing lower concentrations of trehalose.

### Example 4: Stability of Further OPT-302 Formulations

Formulations were prepared commencing with OPT-302 in 40 mM NaCl, 10 mM phosphate pH 7.2, concentrated to 54.5 mg/mL using a 50 kDa membrane.

OPT-302 was purified using a Superdex S200 column loaded at ˜3.2% of the column volume (CV) and run at 0.5 CV/hr in 40 mM NaCl, 10 mM phosphate pH 7.2. S200 fractions with ≥97% monomer were pooled, 7.5% Trehalose spiked into the S200 purified pool with 37.5% Trehalose stock prior to pre-formulation TFF. The % monomer by SE-UPLC was 98.7%

One-fifth of the stock was then spiked to 6.8% trehalose and concentrated to 59 g/L before adding polysorbate 20 (0.03%) and diluted to 40 g/L with 6.85% w/v Trehalose, 0.03% v/v PS20, 10 mM Phos, 40 mM NaCl, pH 7.2.

Three-fifths of the stock was diafiltered with 9.0% w/v Trehalose, 10 mM Phos, pH 7.5, (>7DV) and then split into three portions. The first portion was diluted to 40 mg/ml with 9.0% w/v Trehalose, 10 mM Phosphate pH 7.5. The second portion was spiked to 13.6% trehalose using 40% trehalose, and then diluted to 40 mg/mL OPT-302 to achieve 13.6% w/v Trehalose, 10 mM Phos, pH 7.5, before adding PS-20 (0.01%). The third portion was spiked to 18.1% trehalose using 40% trehalose, and then diluted to 40 mg/mL OPT-302 to achieve 18.1% w/v Trehalose, 10 mM Phos, pH 7.5, before adding PS-20 (0.01%).

The final one-fifth of the stock was diafiltered with 13.6% w/v Trehalose, 20 mM Tris, pH 8.0, (>7DV), diluted to 40 mg/mL OPT-302 in the same buffer before adding PS-20 (0.01%).

SE-UPLC was then performed on all formulations, with the results being shown in the table below.

| | | SE-UPLC |
|---|---|---|
| Formulation no. | Contents | Monomer % |
| Pre-UFDF | 40 mM NaCl, 10 mM Phosphate, | 98.7% |
| stock | pH 7.2 | |
| 1 | 40 mM NaCl, 10 mM Phosphate, | 95.5% |
| | 6.8% w/v Trehalose, 0.03% v/v | |
| | PS20, pH 7.2 | |
| 2 | 10 mM Phosphate, 9.0% w/v | 97.5% |
| | Trehalose, 0.01% v/v PS20, pH | |
| | 7.5 | |
| 3 | 10 mM Phosphate, 13.6% w/v | 98.2% |
| | Trehalose, 0.01% v/v PS20, pH | |
| | 7.5 | |
| 4 | 10 mM Phosphate, 18.1% w/V | 98.3% |
| | Trehalose, 0.01% v/v PS20, pH | |
| | 7.5 | |
| 5 | 20 mM Tris, 13.6% w/V | 98.1% |
| | Trehalose, 0.01% v/v PS20, pH 8 | |

As can be seen from the table, the formulation which was concentrated in the presence of sodium chloride had the lowest monomer stability. Those formulations which underwent diafiltration to remove sodium chloride had improved stability, with % monomer also being higher for those formulations containing higher amounts of trehalose.

### Example 5: Long Term and Accelerated Stability Study

OPT-302 formulations were prepared as follows, all containing 40 mg/mL OPT-302:
1. Lyophilised formulation containing 6.8% w/v trehalose (prepared using 7.5% w/v trehalose dihydrate), 0.03% w/v PS 20, 10 mM sodium phosphate, 40 mM NaCl, pH 7.2.
2. Aqueous formulation containing 6.8% w/v trehalose (prepared using 7.5% w/v trehalose dihydrate), 0.03% PS 20, 10 mM sodium phosphate, 40 mM NaCl, pH 7.2.
3. Aqueous formulation containing 9.0% w/v trehalose (prepared using 10% w/v trehalose dihydrate), 0.01% PS 20, 10 mM sodium phosphate, pH 7.5.
4. Aqueous formulation containing 13.6% w/v trehalose (prepared using 15% w/v trehalose dihydrate), 0.01% PS 20, 10 mM sodium phosphate, pH 7.5.
5. Aqueous formulation containing 18.1% w/v trehalose (prepared using 20% w/v trehalose dihydrate), 0.01% PS 20, 10 mM sodium phosphate, pH 7.5.
6. Aqueous formulation containing 13.6% w/v trehalose (prepared using 15% w/v trehalose dihydrate), 0.01% PS 20, 20 mM Tris, pH 8.

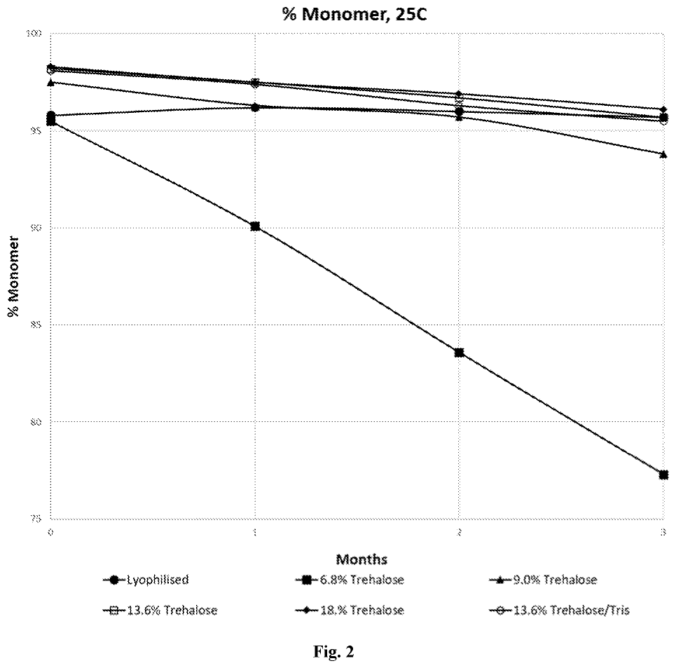

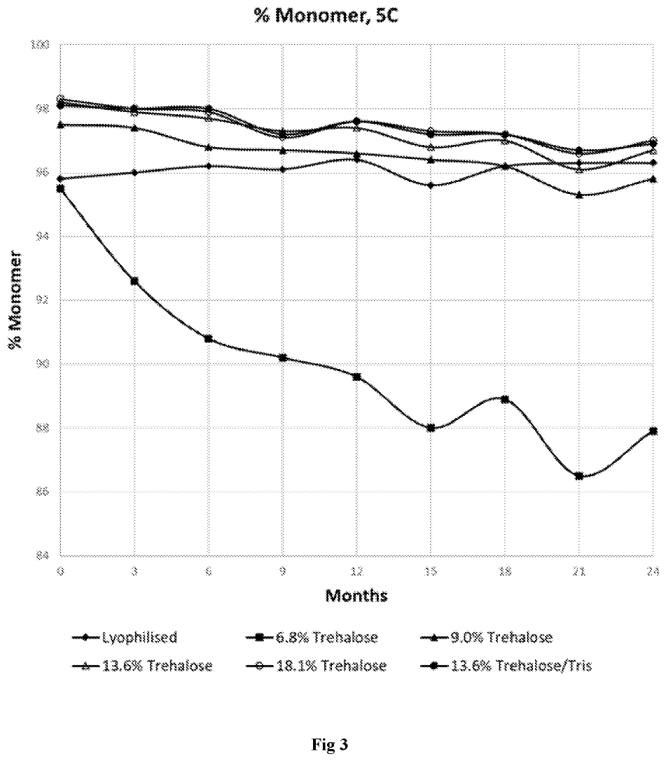

The formulations were set-up at 25° C. (testing monthly for 3 months, SEC), and 5° C. (testing quarterly for 24 months by SEC). Summary results for SEC are provided in FIGS. 2 and 3.

The accelerated results at 25° C. indicate that trehalose concentrations of more than 6.8% w/v substantially stabilize monomer content, and demonstrate that formulations containing high trehalose concentrations without added sodium chloride are likely to be stable at 5° C.

For the real time conditions (5° C., 24 months storage), monomer content for formulations containing more than 6.8% w/v trehalose at 24 months all have significantly greater stability than the comparator formulation.

In summary, in liquid format, long term refrigerated storage (15 month) and accelerated stability results demonstrate that OPT-302 can be reformulated at higher trehalose concentrations which results in excellent stability.

### Example 6: OPT-302 Formulation

A preferred aqueous formulation for OPT-302 was identified, containing the following constituents:

| Constituent | Amount |
|---|---|
| OPT-302 (active ingredient) | 36-44 mg/mL (preferably 40 |
| | mg/mL) |
| Trehalose dihydrate | 10.9% w/v trehalose |
| Sodium phosphate | 10 mM |
| (1:7.8 weight ratio mixture of sodium | |
| dihydrogen phosphate dihydrate and di- | |
| sodium hydrogen phosphate. 7H₂O) | |
| Polyoxyethylene (20) sorbitan monolaurate | 0.01% w/v |
| (Polysorbate 20, Tween 20) | |
| Water For Injection | To volume |
| pH | 7.5 |

Density of formulation=1.041 kg/L

A further comparative OPT-302 formulation has the following constituents:

| Constituent | Amount |
|---|---|
| OPT-302 (active ingredient) | 36-44 mg/mL (preferably |
| | 40 mg/mL) |
| Trehalose dihydrate | 6.8% w/v trehalose |
| | (7.5% w/v trehalose |
| | dihydrate) |
| Sodium phosphate | 10 mM |
| (Mixture of sodium dihydrogen phosphate | |
| dihydrate and di-sodium hydrogen | |
| phosphate. 7H₂O) | |
| Sodium chloride | 40 mM |
| Polyoxyethylene (20) sorbitan monolaurate | 0.03% w/v |
| (Polysorbate 20, Tween 20) | |
| Water For Injection | To volume |
| pH | 7.2 |

The comparative formulation was found to form high levels of OPT-302 dimer on storage, as demonstrated by Example 8 below.

### Example 7: Preparation of OPT-302 Formulation

To a virus-filtered pool containing OPT-302 (Virus Filtered Pool) is added an aliquot of aqueous solution containing trehalose at high concentration (37.5% w/v trehalose dihydrate, 10 mM sodium phosphate, pH 7.5) (Trehalose Spike) in order to achieve a concentration of 12% w/v trehalose dihydrate and mixed for at least 10 minutes to provide a mixture (the Adjusted Virus Filtered Pool).

The mixture is filtered through a 0.2 μm filter and subjected to ultrafiltration-diafiltration and concentration using tangential flow filtration, using an Equilibration, Diafiltration and Flush Buffer containing 12% w/v trehalose dihydrate, 10 mM sodium phosphate at pH 7.5, to provide a mixture having a target OPT-302 concentration of 46-55 mg/mL (UFDF Pool).

The concentration of OPT-302 in the UFDF Pool is determined, and an amount of Dilution Buffer (12% w/v trehalose dihydrate, 10 mM sodium phosphate, pH 7.5) is added to the UFDF Pool to obtain an OPT-302 concentration of around 44 g/L (Diluted UFDF Pool). The Diluted UFDF Pool is mixed using a Wave Mixer for at least 30 minutes.

The target weight of Formulation Buffer (12% w/v trehalose dihydrate, 10 mM sodium phosphate, polyoxyethylene (20) sorbitan monolaurate, pH 7.5) required to be added to the diluted UFDF Pool to obtain a concentration of polyoxyethylene (20) sorbitan monolaurate of 0.01% (w/v) is calculated and added. The formulated, diluted UFDF Pool is mixed using a wave mixer for at least 15 minutes.

The expected concentration of OPT-302 is in the range of from 36-44 mg/mL.

### Example 8: Stability of OPT-302 Formulations

Three OPT-302 formulations were prepared having the following constituents:
Formulation 1: 41.5 mg/mL OPT-302, 10 mM sodium phosphate, 12% w/v trehalose dihydrate (10.9% w/v trehalose), 0.01% w/v polyoxyethylene (20) sorbitan monolaurate, WFI, pH 7.5.
Comparative Formulation 1: 41 mg/mL OPT-302, 10 mM sodium phosphate, 40 mM sodium chloride, 7.5% w/v trehalose dihydrate (6.8% w/v trehalose), 0.03% w/v polyoxyethylene (2) sorbitan monolaurate, WFI, pH 7.2;
Comparative Formulation 2: 42.4 mg/mL OPT-302, 10 mM sodium phosphate, 40 mM sodium chloride, 7.5% w/v trehalose dihydrate (6.8% w/v trehalose), 0.03% w/v polyoxyethylene (2) sorbitan monolaurate, pH 7.2, WFI;

The formulations were stored at various temperatures, and stability properties of the formulations were determined. Data for Formulation 1 stored at −20, 5, 25 and 40° C. is presented below, together with data for the Comparative Formulations stored at 25 and 40° C.

### Formulation 1 at −20° C.

| Criteria | 0 months | 0.5 months | 1 month | 2 months |
|---|---|---|---|---|
| Appearance | Clear | Very slightly | Very slightly | Very slightly |
| Visual | Slightly | opalescent | opalescent | opalescent |
| inspection | yellowish | Slightly | Slightly | Slightly |
| (Colour, | liquid | yellowish | yellowish | yellowish |
| clarity and | No particles | liquid | liquid | liquid |
| visible | | No particles | No particles | No particles |
| particles) | | | | |
| Purity | Monomer: | Monomer: | Monomer: | Monomer: |
| SEC-UPLC | 99% | 98% | 99% | 98% |
| | High MWT: | High MWT: | High MWT: | High MWT: |
| | 1% | 1% | 1% | 1% |
| | Low MWT: | Low MWT: | Low MWT: | Low MWT: |
| | 0% | 0% | 0% | 0% |
| Con- | 41 mg/mL | 41 mg/mL | 42 mg/mL | 42 mg/mL |
| centration | | | | |
| OD280 | | | | |

### Formulation 1 at 5° C.

| Criteria | 0 months | 0.5 months | 1 month | 2 months |
|---|---|---|---|---|
| Appearance | Clear | Very slightly | Very slightly | Very slightly |
| Visual | Slightly | opalescent | opalescent | opalescent |
| inspection | yellowish | Slightly | Slightly | Slightly |
| (Colour, | liquid | yellowish | yellowish | yellowish |
| clarity and | No particles | liquid | liquid | liquid |
| visible | | No particles | No particles | No particles |
| particles) | | | | |
| Purity | Monomer: | Monomer: | Monomer: | Monomer: |
| SEC- | 99% | 98% | 98% | 98% |
| UPLC | High MWT: | High MWT: | High MWT: | High MWT: |
| | 1% | 1% | 2% | 2% |
| | Low MWT: | Low MWT: | Low MWT: | Low MWT: |
| | 0% | 0% | 0% | 0% |
| Con- | 41 mg/mL | 42 mg/mL | 41 mg/mL | 42 mg/mL |
| centration | | | | |
| OD280 | | | | |

### Formulation 1 at 25° C.

| Criteria | 0 months | 0.5 months | 1 month | 2 months |
|---|---|---|---|---|
| Appearance | Clear | Very slightly | Very slightly | Very slightly |
| Visual | Slightly | opalescent | opalescent | opalescent |
| inspection | yellowish | Slightly | Slightly | Slightly |
| (Colour, | liquid | yellowish | yellowish | yellowish |
| clarity and | No particles | liquid | liquid | liquid |
| visible | | No particles | No particles | No particles |
| particles) | | | | |
| Purity | Monomer: | Monomer: | Monomer: | Monomer: |
| SEC- | 99% | 98% | 98% | 97% |
| UPLC | High MWT: | High MWT: | High MWT: | High MWT: |
| | 1% | 2% | 2% | 2% |
| | Low MWT: | Low MWT: | Low MWT: | Low MWT: |
| | 0% | 0% | 0% | 1% |
| Concentration | 41 mg/mL | 43 mg/mL | 41 mg/mL | 42 mg/mL |
| OD280 | | | | |

### Formulation 1 at 40° C.

| Criteria | 0 months | 0.5 months | 1 month |
|---|---|---|---|
| Appearance | Clear | Very slightly | Very slightly |
| Visual inspection | Slightly | opalescent | opalescent |
| (Colour, | yellowish | Slightly | Slightly |
| clarity and | liquid | yellowish | yellowish |
| visible particles) | No particles | liquid | liquid |
| | | One extrinsic | No particles |
| | | particle | |
| Purity | Monomer: 99% | Monomer: 92% | Monomer: 88% |
| SEC-UPLC | High MWT: 1% | High MWT: 6% | High MWT: 9% |
| | Low MWT: 0% | Low MWT: 2% | Low MWT: 3% |
| Concentration | 41 mg/mL | 41 mg/mL | 42 mg/mL |
| OD280 | | | |

### Comparative Formulation 1 at 25° C.

| Criteria | 0 months | 0.5 months | 1 month | 2 months |
|---|---|---|---|---|
| Appearance | Very slightly | Very slightly | Very slightly | Very slightly |
| Visual | opalescent | opalescent | opalescent | opalescent |
| inspection | Slightly | Slightly | Slightly | Slightly |
| (Colour, | yellowish | yellowish | yellowish | yellowish |
| clarity and | liquid | liquid | liquid | liquid |
| visible | Few particles | No particles | No particles | No particles |
| particles) | | | | |
| Purity | Monomer: | Monomer: | Monomer: | Monomer: |
| SEC- | 95% | 93% | 90% | 86% |
| UPLC | High MWT: | High MWT: | High MWT: | High MWT: |
| | 5% | 6% | 9% | 13% |
| | Low MWT: | Low MWT: | Low MWT: | Low MWT: |
| | 0% | 0% | 1% | 1% |
| Con- | 43 mg/mL | 42 mg/mL | 43 mg/mL | 43 mg/mL |
| centration | | | | |
| OD280 | | | | |

### Comparative Formulation 1 at 40° C.

| Criteria | 0 months | 0.5 months | 1 month |
|---|---|---|---|
| Appearance | Very slightly | Very slightly | Very slightly |
| Visual inspection | opalescent | opalescent | opalescent |
| (Colour, | Slightly | Slightly | Slightly |
| clarity and | yellowish | yellowish | yellowish |
| visible particles) | liquid | liquid | liquid |
| | Few particles | No particles | No particles |
| Purity | Monomer: 95% | Monomer: 69% | Monomer: 65% |
| SEC-UPLC | High MWT: 5% | High MWT: 29% | High MWT: 33% |
| | Low MWT: 0% | Low MWT: 2% | Low MWT: 3% |
| Concentration | 43 mg/mL | 42 mg/mL | 44 mg/mL |
| OD280 | | | |

### Comparative Formulation 2 at 25° C.

| Criteria | 0 months | 0.5 months | 1 month | 2 months |
|---|---|---|---|---|
| Appearance | Clear | Very slightly | Very slightly | Very slightly |
| | Slightly | opalescent | opalescent | opalescent |
| | yellowish | | | |
| | liquid | | | |
| Visual | No particles | Slightly | Slightly | Slightly |
| inspection | | yellowish | yellowish | yellowish |
| (Colour, | | liquid | liquid | liquid |
| clarity and | | No particles | No particles | No particles |
| visible | | | | |
| particles) | | | | |
| Purity | Monomer: | Monomer: | Monomer: | Monomer: |
| SEC-UPLC | 97% | 95% | 93% | 89% |
| | High MWT: | High MWT: | High MWT: | High MWT: |
| | 3% | 5% | 6% | 10% |
| | Low MWT: | Low MWT: | Low MWT: | Low MWT: |
| | 0% | 0% | 0% | 1% |
| Con- | 41 mg/mL | 40 mg/mL | 39 mg/mL | 43 mg/mL |
| centration | | | | |
| OD280 | | | | |

### Comparative Formulation 2 at 40° C.

| | | | |
|---|---|---|---|
| Criteria | 0 months | 0.5 months | 1 month |
| Appearance | Clear | Very slightly | Very slightly |
| Visual inspection | Slightly | opalescent | opalescent |
| (Colour, | yellowish | Slightly | Slightly |
| clarity and | liquid | yellowish | yellowish |
| visible particles) | No particles | liquid | liquid |
| | | No particles | No particles |
| Purity | Monomer: 97% | Monomer: 73% | Monomer: 67% |
| SEC-UPLC | High MWT: 3% | High MWT: 26% | High MWT: 30% |
| | Low MWT: 0% | Low MWT: 2% | Low MWT: 2% |
| Concentration | 41 mg/mL | 40 mg/mL | 38 mg/mL |
| OD280 | | | |

Formulation 1 had lower levels of high molecular weight species formation over time, compared with comparative formulations 1 and 2, particularly at higher temperature conditions.

Formulation 1 was also demonstrated to have good activity in respect of binding to VEGF-C and VEGF-D, as determined by ELISA.

### ELISA Method

The assay plate was coated overnight with VEGF-C ligand at 0.1 μg/mL or VEGF-D ligand at 1.0 μg/mL. OPT-302 reference standard and test samples were diluted to a starting concentration of 1500 ng/ml and serial 2-fold dilutions are performed covering from 1.5 ng/mL to 1500 ng/mL. The plate was incubated for 60 min at 25° C. and then washed to remove unbound sample. The bound molecules were detected by adding HRP conjugated rabbit anti-human IgG to the assay plate and incubated for 60 min at 25° C. This was followed by the addition of TMB substrate and the assay plates were incubated in the dark for 10 minutes at room temperature. Colour development was stopped by adding 1M HCl stop solution, which was detected by absorbance at 450 nm. The intensity of the yellow colour was proportional to the amount of bound OPT-302 molecules, which in turn reflected the activity of the OPT-302 reference standard or test samples. 4-parameter curves were then generated by plotting the mean values against log10 of the serial 2-fold dilution concentrations. The reportable value was relative potency (%) of the test sample, i.e. the ratio of the EC50 of the reference standard to the EC50 of the test sample.

On storage at −20° C., Formulation 1 was found to have the following activity: 0 months, VEGF-C: 107%, VEGF-D: 102%; 0.5 months, VEGF-C 126%, VEGF-D 116%; 1 month, VEGF-C: 118%, VEGF-D: 100%; 2 months, VEGF-C: 108%, VEGF-D: 106%.

On storage at 5° C., Formulation 1 was found to have the following activity: 0 months, VEGF-C: 107%, VEGF-D: 102%; 0.5 months, VEGF-C 114%, VEGF-D 112%; 1 month, VEGF-C: 115%, VEGF-D: 101%; 2 months, VEGF-C: 107%, VEGF-D: 105%.

### Example 9: Stability of OPT-302 Formulations

A batch of a preferred aqueous formulation for OPT-302 was prepared, containing the following constituents:

| Constituent | Amount |
|---|---|
| OPT-302 (active ingredient) | 36-44 mg/ml |
| Trehalose dihydrate | 10.9% w/v trehalose |
| Sodium phosphate | 10 mM |
| (1:7.8 weight ratio mixture of sodium | |
| dihydrogen phosphate dihydrate and di- | |
| sodium hydrogen phosphate.7H₂O) | |
| Polyoxyethylene (20) sorbitan monolaurate | 0.01% w/V |
| (Polysorbate 20, Tween 20) | |
| Water For Injection | To volume |
| pH | 7.5 |

Portions of the formulation batch were stored at −20±5° C. and 5±3° C. for prolonged periods of time, and stability properties of the formulations were determined.

When stored at either −20° C. or 5° C. for up to 24 months, key stability assays such as SE-UPLC showed minimal changes in monomer content, declining from the initial amount by 0.8% at −20° C., and by 3% at 5° C., while high-molecular weight (dimer) increased by 0.7% at −20° C., and by 2.2% at 5° C. compared to initiation of the stability study. Similarly, binding activity measured by ELISA decreased minimally by 9%-19% for VEGF-C and VEGF-D.

The formulation had excellent storage stability properties at −20 and 5° C. over the specified time periods.

A further batch of OPT-302 formulated as described for the batch above, was similarly subjected to 24 months storage at 5±3° C., followed by analysis by mass spectrometry to characterise isoform variants. It was found that deamidation variants increased by 9% over the period, isomerised variants decreased by 9%, while there were negligible changes in oxidised variants. Therefore, charged variant changes were overall minimal across long-term (24 months) storage, indicating preservation of molecular structure in the formulation.

1. An aqueous pharmaceutical composition, comprising:
an active agent which is a soluble VEGFR-3 trap molecule, the active agent being present at a concentration in the range of from 5 mg/mL to 250 mg/mL, wherein the VEGFR-3 trap molecule is or comprises a polypeptide;
trehalose;
a buffer; and
water;
wherein the pH of the aqueous pharmaceutical composition is in the range of from 6.5 to 8.0;
and wherein the pharmaceutical composition does not contain added sodium chloride.

2. (canceled)

3. An aqueous pharmaceutical composition as claimed in claim 1, wherein one of the following applies:
i) the pharmaceutical composition comprises trehalose in a concentration of at least 7.0% w/v or at a concentration of from 8.5% w/v to 15% w/v, or the composition comprises about 10.9% w/v trehalose; or
ii) the pharmaceutical composition comprises trehalose at a concentration of up to 20% w/v.

4-6. (canceled)

7. An aqueous pharmaceutical composition as claimed in claim 1, wherein the soluble VEGFR-3 trap molecule comprises a ligand binding polypeptide fused to an immunoglobulin constant domain fragment, the ligand binding polypeptide comprising immunoglobulin-like domains 1-3 of the extracellular domain of human VEGFR-3 and optionally having one or more modifications in an N-glycan region of the extracellular domain.

8. An aqueous pharmaceutical composition as claimed in claim 7, wherein one of the following applies:
i) the ligand binding polypeptide comprises the amino acid sequence defined by positions 25-329 of SEQ ID NO: 1, with the proviso that positions of the polypeptide corresponding to positions 104-106 of SEQ ID NO: 1 are not identical to N-X-S or N-X-T; wherein the ligand binding polypeptide retains four N-glycosylation sequon sites corresponding to positions 33-35 of SEQ ID NO: 1, positions 166-168 of SEQ ID NO: 1, positions 251-253 of SEQ ID NO: 1, and positions 299-301 of SEQ ID NO: 1, and is glycosylated at said four N-glycosylation sequon sites;
i) the immunoglobulin constant domain fragment comprises the amino acid sequence defined by positions 99-330 of SEQ ID NO: 2;
iii) the ligand binding polypeptide comprises the amino acid sequence defined by positions 25-329 of SEQ ID NO: 1; wherein the ligand binding polypeptide retains five N-glycosylation sequon sites corresponding to positions 33-35 of SEQ ID NO: 1, positions 104-106 of SEQ ID NO: 1, positions 166-168 of SEQ ID NO: 1, positions 251-253 of SEQ ID NO: 1, and positions 299-301 of SEQ ID NO: 1, and is glycosylated at said five N-glycosylation sequon sites; or
iv) the immunoglobulin constant domain fragment comprises the amino acid sequence defined by positions 99-330 of SEQ ID NO: 2, optionally wherein the soluble VEGFR-3 trap molecule has the amino acid sequence set forth in SEQ ID NO: 7, or has an amino acid sequence as defined by positions 1-547 of SEQ ID NO: 7.

9-13. (canceled)

14. An aqueous pharmaceutical composition as claimed in claim 1, wherein one of the following applies:
i) the active agent is present at a concentration of up to 120 mg/ml; or
ii) the active agent is present at a concentration of about 40 mg/mL, or about 80 mg/mL, or about 120 mg/mL.

15. (canceled)

16. An aqueous pharmaceutical composition as claimed in claim 1, wherein the pH of the composition is in the range of from 7.2 to 7.8, or wherein the pH of the composition is about 7.5.

17. (canceled)

18. An aqueous pharmaceutical composition as claimed in claim 1, wherein one of the following applies:
i) the buffer is a sodium phosphate;
ii) the buffer is present in a concentration in the range of from 5 mM to 100 mM; or
the buffer is present in a concentration in the range of up to 50 mM, optionally wherein the buffer is present in a concentration of about 10 mM.

19-21. (canceled)

22. An aqueous pharmaceutical composition as claimed in claim 1, wherein the composition comprises a surfactant, and optionally one of the following applies:
i) the surfactant is polyoxyethylene (20) sorbitan monolaurate or polyoxyethylene (20) sorbitan monooleate; or
ii) surfactant is present at a concentration in the range of from 0.005% to 0.2% w/v, or the surfactant is present at a concentration of about 0.01% w/v.

23-25. (canceled)

26. An aqueous pharmaceutical composition as claimed in claim 1, wherein the composition has an osmolality in the range of from 300 mOsm/kg to 1000 mOsm/kg, or wherein the composition has an osmolality in the range of from 400 mOsm/kg to 600 mOsm/kg.

27-29. (canceled)

30. An aqueous pharmaceutical composition as claimed in claim 1, wherein one of the following applies:
i) the composition is substantially free of sodium chloride;
ii) the composition does not contain an additional sugar; or
iii) the composition does not contain an additional tonicity modifier.

31-32. (canceled)

33. An aqueous pharmaceutical composition as claimed in claim 1, wherein the composition essentially consists of:
an active agent in a concentration of about 40 mg/ml, which is a soluble VEGFR-3 trap molecule that comprises a ligand binding polypeptide fused to an immunoglobulin constant domain fragment, the ligand binding polypeptide comprising immunoglobulin-like domains 1-3 of the extracellular domain of human VEGFR-3 and optionally having one or more modifications in an N-glycan region of the extracellular domain;
trehalose in a concentration of about 10.9% w/v;
sodium phosphate in a concentration of about 10 mM;
polyoxyethylene (20) sorbitan monolaurate in a concentration of about 0.01% w/v; and
water;
wherein the pH of the aqueous pharmaceutical composition is about 7.5.

34. A lyophilised pharmaceutical composition for reconstitution, comprising:
an active agent which is a soluble VEGFR-3 trap molecule, wherein the VEGFR-3 trap molecule is or comprises a polypeptide;
trehalose; and
a buffer;
wherein the weight ratio of trehalose to active agent is in the range of from 1:3 to 40:1.

35. A lyophilised pharmaceutical composition as claimed in claim 34, wherein the weight ratio of trehalose to active agent is in the range of from 1:1 to 7.5:1, from 1:1 to 5:1, or from 2.1:1 to 4.5:1, or wherein the weight ratio of trehalose to active agent is about 2.7:1.

36. (canceled)

37. A lyophilised pharmaceutical composition as claimed in claim 34, wherein the buffer is a sodium phosphate, optionally wherein the weight ratio of sodium phosphate to active agent is in the range of from 1:3 to 1:1000, or from 1:3 to 1:200, or from 1:5 to 1:100, or wherein the weight ratio of sodium phosphate to active agent is about 0.03:1.

38-39. (canceled)

40. A lyophilised pharmaceutical composition as claimed in claim 34, wherein the composition comprises a surfactant, optionally wherein the surfactant is polyoxyethylene (20) sorbitan monolaurate or polyoxyethylene (20) sorbitan monooleate.

41-42. (canceled)

43. A pharmaceutical composition as claimed in claim 1, wherein the pharmaceutical composition is formulated for intravitreal injection.

44. (canceled)

45. A method of treating and/or preventing a disease or disorder associated with aberrant neovascularisation, angiogenesis and/or lymphangiogenesis in a subject, comprising administering to the subject an effective amount of a pharmaceutical composition as claimed in claim 1, optionally wherein the disease or disorder is an ocular disease or disorder.

46-48. (canceled)

49. A method as claimed in claim 45, wherein the ocular disease or disorder is selected from the group consisting of macular degeneration, diabetic retinopathy, macular edema, retinal vein occlusion and macular telangiectasia.

50.-51. (canceled)

52. A method as claimed in claim 45, wherein the pharmaceutical composition is administered in combination with a further active agent, optionally wherein one of the following applies:
i) the further active agent is an anti-VEGF-A agent or an anti-VEGF-B agent; and
ii) the further active agent is selected from the group consisting of ranibizumab, aflibercept, bevacizumab and brolucizumab.

53.-54. (canceled)

55. A method as claimed in claim 45, wherein the pharmaceutical composition is administered intravitreally, or wherein the pharmaceutical composition is administered using a port device which is implanted in an eye, which port device comprises a reservoir for the pharmaceutical composition, and permits controlled release of active agent into the vitreous of the eye.

56.-59. (canceled)


SEQUENCE LISTING (8 sequences)

SEQ ID NO: 1 - protein, 764 aa, Homo sapiens
MQRGAALCLRLWLCLGLLDGLVSGYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGRYLCSVCNAKGCVNSSASVA

SEQ ID NO: 2 - protein, 330 aa, Homo sapiens [sequence omitted]

SEQ ID NO: 3 - protein, 537 aa, synthetic construct
  CARBOHYD 9..11: note = N-Glycosylation
  CARBOHYD 142..144: note = N-Glycosylation
  CARBOHYD 227..229: note = N-Glycosylation
  CARBOHYD 275..277: note = N-Glycosylation
  CARBOHYD 387..389: note = N-Glycosylation
  DOMAIN 1..305: note = VEGFR-3 ECD 1-3 Portion
  DOMAIN 306..537: note = Fc Portion
YSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 537 aa, synthetic construct
  CARBOHYD 9..11: note = N-Glycosylation
  CARBOHYD 142..144: note = N-Glycosylation
  CARBOHYD 227..229: note = N-Glycosylation
  CARBOHYD 275..277: note = N-Glycosylation
  CARBOHYD 387..389: note = N-Glycosylation
  DOMAIN 1..305: note = VEGFR-3 ECD 1-3 Portion
  DOMAIN 306..537: note = Fc Portion
  VAR_SEQ 310: note = Substitution of C with S
YSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 547 aa, synthetic construct
  CARBOHYD 9..11: note = N-Glycosylation
  CARBOHYD 142..144: note = N-Glycosylation
  CARBOHYD 227..229: note = N-Glycosylation
  CARBOHYD 275..277: note = N-Glycosylation
  CARBOHYD 397..399: note = N-Glycosylation
  DOMAIN 1..305: note = VEGFR-3 ECD 1-3 Portion
  DOMAIN 316..547: note = Fc Portion
YSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 547 aa, synthetic construct
  CARBOHYD 9..11: note = N-Glycosylation
  CARBOHYD 142..144: note = N-Glycosylation
  CARBOHYD 227..229: note = N-Glycosylation
  CARBOHYD 275..277: note = N-Glycosylation
  CARBOHYD 397..399: note = N-Glycosylation
  DOMAIN 1..305: note = VEGFR-3 ECD 1-3 Portion
  DOMAIN 316..547: note = Fc Portion
  VAR_SEQ 320: note = Substitution of C with S
YSMTPPTLNITEESHVIDTGDSLSISCRGQHPLEWAWPGAQEAPATGDKDSEDTGVVRDCEGTDARPYCKVLLLHEVHAQDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 548 aa, synthetic construct
  CARBOHYD 9..11: note = N-Glycosylation
  CARBOHYD 80..82: note = N-Glycosylation
  CARBOHYD 142..144: note = N-Glycosylation
  CARBOHYD 227..229: note = N-Glycosylation
  CARBOHYD 275..277: note = N-Glycosylation
  CARBOHYD 398..400: note = N-Glycosylation
  DOMAIN 1..305: note = VEGFR-3 ECD 1-3 Portion
  DOMAIN 306..316: note = Factor Xa linker
  DOMAIN 317..548: note = Fc Portion
YSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHEDPIEGRGGGGGDPKSCDKPHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 8 - protein, 11 aa, synthetic construct
DPIEGRGGGGG